(19) United States
(12) Patent Application Publication
(10) Pub. No.: US 2021/0268240 A1
(43) Pub. Date: Sep. 2, 2021

(54) DEVICE AND METHODS FOR THERAPEUTIC ADMINSTRATION USING A BUTTERFLY ASSEMBLY AND INFUSION DRIVER

(71) Applicant: INNOVATIVE HEALTH SCIENCES, LLC, Chester, NY (US)

(72) Inventor: Andrew SEALFON, Chester, NY (US)

(73) Assignee: INNOVATIVE HEALTH SCIENCES, LLC, Chester, NY (US)

(21) Appl. No.: 17/200,294

(22) Filed: Mar. 12, 2021

Related U.S. Application Data
(63) Continuation-in-part of application No. 17/026,050, filed on Sep. 18, 2020.
(60) Provisional application No. 62/902,591, filed on Sep. 19, 2019.

(51) Int. Cl. A61M 25/06; A61M 5/145

(52) U.S. Cl. CPC A61M 25/0637; A61M 25/0618; A61M 2205/581; A61M 2005/14506; A61M 2205/582; A61M 5/145

(57) ABSTRACT

Butterfly assemblies to deliver a fluid into a patient's anatomic space include a body and extensions. The body has an opening to receive a needle to penetrate into a patient's anatomic space, and the extensions are hinged to the body and movable between a closed, compact position and an expanded, open position without significant biasing forces tending to move the extensions into either position. Each extension has an outer periphery at least partially surrounding an interior surface having a locking structure to mate with the other extension and lock the extensions together in the closed, compact position.

### CROSS REFERENCE TO RELATED APPLICATION

This application is a continuation-in-part of U.S. patent application Ser. No. 17/026,050 filed on Sep. 18, 2020, which claims the benefit of U.S. Provisional Application No. 62/902,591 filed on Sep. 19, 2019. This application incorporates by reference the entire contents of U.S. patent application Ser. No. 17/026,050 filed on Sep. 18, 2020, and U.S. Provisional Application No. 62/902,591 filed on Sep. 19, 2019.

### TECHNICAL FIELD

The invention relates generally to systems and methods for precision matched selectable flow rate controllers and needle sets. More specifically, the invention relates to selectable flow rate controllers, butterfly assemblies, and needle sets to deliver fluids for infusion therapy safely and accurately using a substantially constant pressure syringe driver.

### BACKGROUND

Current infusion systems on the market are mostly electrically powered and function by delivering fluids at a pre-set flow rate. In order to maintain the preset flow rate, the system must increase pressure in response to any blockage or other increase in fluid resistance from anywhere in the infusion circuit. This increased pressure can cause severe site reactions, pain, and tissue necrosis. Other infusion systems consist of mechanical syringe drivers, but these generally require a separate flow rate tubing selection for each desired flow rate, which cannot be easily changed once the infusion begins. Still others have a variable flow rate controller for subcutaneous administrations, but it is not calibrated, leaving the flow rate delivered a mystery and complicating the optimization of the infusion treatment.

Infusion systems and methods of use administer fluids (generally medications in liquid form) including immunoglobulins for Primary Immune Deficiency Diseases (PIDD) or neuromodulation (neurology), monoclonal antibody therapies for various diseases, hydration, antibiotics, analgesia, and other therapies for other diseases. An infusion pump is a medical device that delivers fluids, including nutrients and medications, including immunoglobulin or antibiotics, into a patient in controlled amounts. The nutrients and medications can include insulin, other hormones, antibiotics, chemotherapy drugs, pain relievers, and other fluids.

Infusion pumps can be used to deliver fluids intravenously, as well as subcutaneously (beneath the skin), arterially, and epidurally (within the surface of the central nervous system). Infusion pumps can reliably administer fluids in ways that would be impractically expensive, unsafe, or unreliable if performed manually by a nursing staff. Infusion pumps offer advantages over manual administration of fluids, including the ability to deliver fluids in very small volumes and the ability to deliver fluids at precisely programmed rates or automated intervals. For example, infusion pumps can administer 1 ml per hour injections (too small a dose for drip methods), injections every minute, injections with repeated boluses requested by the patient (e.g., for patient-controlled analgesia up to a maximum allowed number of boluses over a time period), or fluids whose volumes and delivery vary by the time of day.

Mechanical constant pressure infusion pump systems often use disposable infusion sets to link the pump system to an infusion site of a patient. These sets usually have fixed flow rate tubing between the infusion site and the infusion pump. For constant flow electric pump systems, the tubing is referred to as an “extension set” and has undefined flow properties as the electric pump will adjust to the pressure required to maintain the desired flow rate.

As used herein, “needle set” and “intravenous infusion set” are “administration sets” and refers to the delivery assembly of tubing, luer locks, line locks, flow rate controllers, needles, and needle safety features (e.g., butterfly assembly or disc). The “tubing set” refers to the tubing used in the “needle set” and “intravenous infusion set.”

Further, in conventional mechanical infusion systems, separate flow rate restriction tubing is used to create different flow rates for different drugs, intravenous catheters, or subcutaneous needle sets based on the requirements of the infusion rate for the patient. There are currently 22 offerings in the market for precision flow rate tubing sets. Each precision flow rate tubing set includes a set length and a specific diameter provided by the manufacturer. In the case of subcutaneous applications, assuming the same drug is used, each precision flow rate tubing set produces a different flow rate that is dependent upon the number of needle sites used in the needle set, and the diameter and length of tubing and needle used. Subcutaneous needle sets are provided in configurations of 1-8 needles grouped together into a common manifold with each configuration requiring a different series flow rate tubing that may differ in either length and/or diameter. Additionally, in these known systems, there are generally four bore sizes of needles (28 g, 27 g, 26 g, 24 g) which also result in different flow rates with each precision flow rate tubing set. These flow rates are calculated using a flow rate calculator or a mobile app to enter system parameters (e.g., specific fluid viscosity, etc.) to calculate infusion flow rate and time. For intravenous administrations, most of the drugs are low viscosity, and the intravenous catheters do not impair the flow rate accuracy at lower flow rates (<120 ml/hr). Also, mechanical infusion pumps currently on the market target subcutaneous administrations, ignoring the fact that about 80-90% of all infusions are intravenous.

One example of a variable flow rate controller is described in U.S. Patent Application Publication 2016/0256625. The variable flow rate controller replaces the need for multiple fixed flow rate tubing sets, which minimizes stocking issues. However, it was found that the variable flow rate controller was unpredictable with great flow rate inconsistencies and loss of accuracy at both the low-end and high-end settings. Additionally, these controllers had an unrestricted flow rate at the wide-open maximum setting (i.e., the markings do not directly indicate the flow rate). Thus, when using these systems, clinicians have a difficult time predicting or knowing what the actual delivered flow rates are likely to be. As each infusion is unique, it becomes a clinical challenge to know whether any problems during administration exist in the patient or in the variable flow rate controller device. Without an established baseline, it is difficult to diagnose and correct any infusion complications.

In mechanical constant pressure systems, components in direct or indirect contact with the fluid path influence the final flow rate delivered to the patient. Any part of the system can contribute to an incorrect flow rate being delivered to the patient and the associated harmful adverse reactions that can occur to the patient.

While some adverse treatment events may be the result of user error, many of the reported adverse events with previous systems are related to deficiencies in infusion system design and engineering, with the risk usually being an excessive flow rate or high output pressure. The additional calculations required for each variation of needle and tubing sets and controllers adds unneeded complexity and points of error. These deficiencies create problems themselves or contribute to user error by manifesting themselves in improper flow rates of the infusion fluids at the patient infusion sites.

The above information disclosed in this Background section is only for understanding of the background of the inventive concepts, and, therefore, it may contain information that does not constitute prior art.

### SUMMARY

The infusion systems constructed, and the methods performed, according to the principles and exemplary implementations of the invention address one of more the above-noted deficiencies. For example, infusion systems constructed according to the principles and some exemplary implementations of the invention (and methods implementing the same) deliver infusion fluid to a patient using a matched variable flow rate controller and an administration set and a constant pressure syringe driver for delivery of the infusion fluid. In one example implementation of the invention, a precision matched infusion system delivers immunoglobulin for subcutaneous applications. In another example implementation of the invention, a precision matched infusion system uses a constant pressure syringe driver and a matched variable flow rate controller and tubing set to deliver an antibiotic infusion for intravenous applications.

In one exemplary implementation of the invention, a calibrated disposable infusion set is used to ensure that the controller delivers the correct flow rate. By constructing and using a calibrated flow rate controller and compatible parts of the flow circuit, systems in accordance with the invention safely and accurately deliver infusion fluids to the patient.

Infusion systems and methods in accordance with the principles and some exemplary implementations of the invention solve many of the major issues of pharmaceutical drug delivery problems. They can drastically improve safety by limiting pressure to safe values. They are much less labor intensive, as they obviate the need for numerous fixed rate tubing sets. Infusion systems and methods in accordance with the principles and some exemplary implementations of the invention can provide these benefits at a much lower price point and may be scalable for manufacture and thus can meet the demands of new infectious viruses like COVID 19. They can be used by clinicians or trained patients, in a hospital, clinic, or at home. Infusion systems and methods in accordance with the principles and some exemplary implementations of systems and methods in accordance with the invention also can provide direct indication of the flow rate—what you see is what you get—and require no calculations, Excel spread sheets, or long lists of tables for referencing the flow rate output for each situation. They can eliminate the need for a range of different flow rate controls, can be automatically calibrated to provide the correct flow rate indications for any number of needle sites, and eliminate errors while improving the sterile compliance by connecting all infusion components together in one package. Infusion systems and methods in accordance with the principles and some exemplary for subcutaneous delivery of immunoglobulins and intravenous delivery of antibiotics can deliver the maximum flow rates of drugs currently on the market and can meet future demands for even faster flow rates. There are currently no systems available on the market that can provide the flexibility, safety, ease of use, and overall infusion performance at a low-cost price point as with infusion systems and methods in accordance with the principles and some exemplary implementations of with the invention.

For example, matching a variable flow rate controller with either an intravenous or subcutaneous administration set solves many of the problems in the art. Intravenous tubing sets are matched and packaged with a variable flow rate controller as a calibrated infusion set. Similarly, in subcutaneous infusions, a subcutaneous needle set is matched and packaged with a variable flow rate controller as another calibrated infusion set. The matched sets are delivered in sterile packages, and several major advantages over prior systems are realized.

These advantages include fewer items to stock, repeatable and accurate flow control settings, and vastly improved patient and caregiver safety. Infusion systems and methods in accordance with the principles and some exemplary implementations of the invention can provide pre-set maximum flow rates (set at the factory or by the health care provider), the number of needle sets may be matched based on a maximum flow rate setting. This improves patient safety as it obviates prior methods of connecting the controller to a needle set (for subcutaneous applications) and eliminates a source of potential contamination in all applications by reducing the chance of sterility contamination.

To circumvent the inconsistencies and inaccuracies of current market offerings, some exemplary implementations of the invention are specifically calibrated to ensure that the controller delivers the precise flow rate, which is clearly indicated on the controller dial for patients and clinicians. Additionally, since the controller enables patients and/or providers to select various flow rates, the need for additional fixed rate tubing sets (current market offerings) is unnecessary. This enables a tailored infusion experience for each patient according to their treatment regimen.

For subcutaneous applications, the more needle sites used, the greater need for higher flow rates from the variable flow rate controller. For example, if the maximum flow rate value used with a four-needle set was used with a single needle set, the delivered rate to the patient would be excessive and would cause discomfort. Conversely, if the maximum flow rate for a single-needle set is used with a four-needle set, the flow rate per site will be well below the maximum flow rate permitted, and the patient will not be able to receive the treatment in the most time efficient manner. Further, matching flow rate controllers constructed in accordance with some exemplary implementations of the invention can correctly account for flow rates at the extreme settings of the controllers and label the flow rate produced, in ml/hr, with a visual reference, so patients are fully aware of the safe range of flow rates.

Exemplary implementations of the invention can provide specific cost advantages over known systems, such as the variable flow rate controller in the U.S. Patent Application Publication 2016/0256624, by simplifying stocking of the needed variable flow rate controller. This avoids the need to stock multiple different variable flow rate controllers. In addition, there is less labor for the health care provider, as they can provide a single matched package with all components that the patient needs. Additionally, reducing the decision-making process and complications when changing needle sets or tubing sets or variable flow rate controllers greatly reduces user errors.

Butterfly assemblies constructed according to the principles and some exemplary implementations of the invention provide specific patient comfort and safety advantages over known systems. For example, the butterfly assemblies include “no memory” hinges to reduce patient discomfort by preventing biasing of the hinges during use. The locking mechanism of the wings of the butterflies includes protrusions that also reduce patient discomfort by increasing the patient contact surface, which makes the protrusions imperceptible or nearly imperceptible to the patient by diffusing contact forces. The locking mechanism feature provides a single-handed easy-to-use closing technique for users by preventing misalignment and requiring smaller closing forces. Further, the needle protector tapered guiding channel feature provides a safe and secure way to maintain the needle in place at a substantially ninety degree orientation so the patient does not insert a crooked needle into their skin, which can cause significant discomfort and/or injecting the drug into the incorrect tissue depth.

Infusion drivers constructed according to the principles and some exemplary implementations of the invention provide specific safety, ease of use, and build advantages over known systems. For example, the infusion drivers may include a level loading mechanism to reduce pinching and makes the drivers easier to use. The drivers are simplified from known drivers to reduce potential failure points, to prevent accidents with reservoir ejection, and to reduce the size of the infusion driver.

Additional features of the inventive concepts will be set forth in the description which follows, and in part will be apparent from the description, or may be learned by practice of the inventive concepts.

According to one aspect of the invention, an infusion system for delivering an infusion fluid into a patient's anatomic space includes: a controller pre-set to deliver a desired flow rate of infusion fluid; and an administration set matched to the controller, the administration set including a pre-determined number of flow tubes having diameters and lengths selected based upon the desired flow rate and number of infusion sites for a specific infusion fluid treatment.

The administration set may include a needle set to subcutaneously deliver the infusion fluid into the patient's anatomic space, and the needle set further may include a pre-determined number of needles having diameters selected based upon the desired flow rate, a number of infusion sites to subcutaneously deliver the infusion fluid into the patient's anatomic space, and the specific infusion fluid to be delivered.

The infusion system further may include a substantially constant pressure infusion driver to deliver the infusion fluid; and the pre-determined number of needles may be pre-calibrated to deliver a predetermined flow rate of the specific infusion fluid at a predetermined infusion fluid pressure based on the number of needles in the administration set, a flow rate of the flow tubes, and the specific infusion fluid to be delivered.

The number of needles in the administration set may include one to eight.

The controller may be configured to be attached to the flow tubes and pre-set to deliver a pre-set flow rate less than or equal to a maximum flow rate for the specific infusion fluid treatment.

The flow tubes and the needles may be packaged in a single-use package.

The administration set may include an intravenous infusion set to intravenously deliver the infusion fluid into the patient's anatomic space, and the intravenous infusion set further may include a tube to receive the infusion fluid from the infusion driver; and a connector to receive the infusion fluid from the controller and the tube to deliver the infusion fluid to an IV bag or catheter at a predetermined flow rate; and the predetermined flow rate may be selected for the specific infusion fluid at a predetermined infusion fluid pressure, and a flow rate of the tube.

The controller may be configured to be attached to the system and pre-set to deliver a pre-set flow rate less than or equal to a maximum flow rate for the specific infusion fluid treatment.

The connector may include a luer lock connector.

According to another aspect of the invention, an infusion system for delivering an infusion fluid into a patient's anatomic space includes: a pump driver to deliver the infusion fluid into the patient's anatomic space at a substantially constant pressure and a desired flow rate; an administration set to deliver the infusion fluid into a patient's anatomic space, and the administration set includes: a pre-determined number of flow tubes having diameters and lengths selected based upon the desired flow rate, and number of infusion sites for a specific infusion fluid treatment.

The administration set may include a needle set to subcutaneously deliver the infusion fluid into the patient's anatomic space, and the needle set further may include: a pre-determined number of needles having diameters selected based upon the desired flow rate, a number of infusion sites to subcutaneously deliver the infusion fluid into the patient's anatomic space, and the specific infusion fluid.

The pre-determined number of needles may be pre-calibrated to deliver a predetermined flow rate of the specific infusion fluid at a predetermined infusion fluid pressure based on the number of needles in the administration set, a flow rate of the flow tubes, and the specific infusion fluid to be delivered.

The number of needles in the administration set may include one to eight.

The driver may be configured to be attached to the flow tubes and pre-set to deliver a pre-set flow rate less than or equal to a maximum flow rate for the specific infusion fluid treatment.

The flow tubes and the needles may be packaged in a single-use package.

The administration set may include an infusion set to intravenously deliver the infusion fluid into the patient's anatomic space, the administration set further may include: a connector to receive the infusion fluid and to deliver the infusion fluid to an IV bag or catheter at a predetermined flow rate selected for the specific infusion fluid treatment at a predetermined infusion fluid pressure based on a flow rate of the flow tubes; and a flow rate controller to be attached to the connector and pre-set to deliver a pre-set flow rate less than or equal to a maximum flow rate for the specific infusion fluid treatment.

The connector may include a luer lock connector.

According to another aspect of the invention, a method of manufacturing an infusion system for delivering a specific infusion fluid to a patient's anatomical space includes the steps of: matching a flow rate controller to an administration set, where the flow controller is pre-set to deliver a desired flow rate of infusion fluid and the administration set includes a predetermined number of flow tubes having lengths and diameters based on the desired flow rate and number of infusion sites for the specific infusion fluid treatment.

The administration set may include a needle set to subcutaneously deliver the infusion fluid into the patient's anatomic space, and the method further may include: selecting a pre-determined number of needles having diameters selected based on the desired flow rate, a number of infusion sites to subcutaneously deliver the infusion fluid into the patient's anatomic space, and the specific infusion fluid.

The method of manufacturing further may include: configuring and pre-calibrating a number of needles to deliver the infusion fluid into the patient's anatomic space, and determining a flow rate of the specific infusion fluid at a pre-determined infusion fluid pressure based on the number of needles in the administration set, a flow rate of the flow tubes, and the specific infusion fluid to be delivered.

The method further may include: configuring the flow rate controller to be attached to the flow tubes; and pre-setting the flow rate controller to deliver a pre-set flow rate less than or equal to a maximum flow rate for the specific infusion fluid treatment.

The method further may include packaging the flow tubes and the needles in a single-use package.

The number of needles of the infusion system may include one to eight.

The infusion system may be configured to intravenously deliver the infusion fluid into the patient's anatomic space, and the method further may include configuring a tube to receive the infusion fluid from an infusion driver; and configuring a connector to receive the infusion fluid from the matched flow rate controller and the tube to deliver the infusion fluid to an IV bag or catheter at a predetermined flow rate selected for the specific infusion fluid treatment at a predetermined infusion fluid pressure based a flow rate of the tube.

The method further may include configuring the flow rate controller to be attached to the connector; and pre-setting the flow rate controller to deliver a pre-set flow rate less than or equal to a maximum flow rate for the specific infusion fluid treatment.

The method further may include providing an infusion driver to deliver the infusion fluid at a substantially constant pressure.

According to another aspect of the invention, an administration set for delivering an infusion fluid into a patient's anatomic space includes a pre-determined number of flow tubes having diameters and lengths selected based upon a desired flow rate of a controller and a number of infusion sites for a specific infusion fluid treatment.

The administration set further may include a controller pre-set to deliver a desired flow rate of infusion fluid, and the administration set may be matched to the controller.

The administration set further may include a pre-determined number of needles having diameters selected based upon the desired flow rate, a number of infusion sites to subcutaneously deliver the infusion fluid into the patient's anatomic space, and the specific infusion fluid to be delivered.

The administration set further may include a tube to receive infusion fluid from a source of infusion fluid; and a connector to receive infusion fluid from the controller and the tube to deliver the infusion fluid to an IV bag or catheter at a predetermined flow rate selected for the specific infusion fluid at a predetermined infusion fluid pressure and a flow rate of the tube.

According to another aspect of the invention, an assembly to deliver a fluid into a patient's anatomic space includes: a body having an opening to receive a needle to penetrate into a patient's anatomic space, and extensions hinged to the body and movable between a closed, compact position and an expanded, open position without any significant biasing force tending to force the extensions into either position, each extension having an outer periphery at least partially surrounding an interior surface having a locking structure to mate with the other extension and lock the extensions together in the closed, compact position.

The interior surfaces of the extensions further may include a guide to prevent misalignment during movement of the extensions to the closed, compact position.

The guide may include a guiding mechanism having a curved protrusion extending from the interior surface of one of the extensions.

The locking structure may be configured to provide sensory feedback to a user to indicate the extensions are in the closed, compact position.

The body may include a hub and the extensions comprise a pair of wings extending from the hub, with the locking structure on one wing may include a projection and the locking structure on the other wing may include a recess to receive the projection.

At least one of the extensions may include a raised surface including a plurality of substantially smooth, spaced projections to increase the surface area of the interior surface and create a diffuse patient contact surface for contacting the patient without causing substantial pain or irritation.

The assembly further may include hinges being in a neutral and substantially un-compressed state when the wings are in the open, expanded position and wings hinges being disposed no more than about 10 degrees from horizontal when the wings are in the open, expanded position.

Each hinge is less than about 0.3 mm thick and about three or less pounds of force may be required to lock the wings into the compact, closed position and about six or more pounds of force may be required to unlock the wings.

The assembly further may include a needle in fluid communication with the body and having an end configured to penetrate into a patient's anatomic space to deliver fluid thereto, a needle protector surrounding the needle, and a groove disposed in at least one of the extensions to guide and maintain the needle protector in the groove before use.

The assembly further may include a ball-and-pivot mechanism to receive a portion of a fluid delivery needle and allow rotation of the needle to reduce forces transmitted to the needle during use of the assembly.

According to another aspect of the invention, an assembly to deliver a fluid into a patient's anatomic space includes: a needle to penetrate into a patient's anatomic space, a needle protector surrounding the needle, a body having a longitudinal axis and an opening to receive the needle, extensions hinged to the body and movable between a closed, compact position and an expanded, open position, each extension having an outer periphery at least partially surrounding an interior surface having a locking structure to mate with the other extension and lock the extensions together in the closed, compact position, and a recess disposed in at least one of the extensions to guide and maintain the needle protector in a substantially 90 degree orientation relative to the longitudinal axis of the body.

According to another aspect of the invention, a method for delivering a fluid into a patient's anatomic space through a needle having a needle protector supported by a butterfly assembly having pair of wings extending from the base, with a guiding channel supporting the needle protector in substantially 90 degrees relative to a longitudinal axis of the base, the wings being movable between a compact, closed position surrounding the needle and an open, expanded position exposing the needle for insertion into the patient's anatomic space, the method includes: moving the butterfly assembly into the open, expanded position exposing the needle and the needle protector, removing the needle protector, inserting the needle into a patient's anatomic space, and pressing the pair of wings against the patient without any significant biasing force tending to force wings into the closed position.

The method further may include delivering a therapeutic treatment fluid into the patient's anatomic space via the needle.

Pressing the pair of wings against the patient may include presenting a raised surface on the wings to the patient to increase the surface area of wings and creates a diffuse patient contact surface for contacting the patient without causing substantial pain or irritation.

Moving the butterfly assembly into the open, expanded position may include moving the wings about a hinge less than about 0.3 mm thick to the open, expanded position before pressing the wings against the patient.

The method further may include withdrawing the needle from the patient, guiding the wings into the closed position with a curved protrusion extending from an interior surface of one of the wings, and locking the wings in the closed, compact position with a locking mechanism disposed on interior surfaces of the wings.

The method further may include providing sensory feedback to indicate the wings are in the closed, compact position.

About three or less pounds of force may be required to lock the wings into the compact, closed position and about six or more pounds of force may be required to unlock the wings.

According to another aspect of the invention, an infusion assembly for delivery of an infusion fluid into a patient's anatomic space at a substantially constant pressure, the infusion assembly includes: a reservoir for the infusion fluid, and a driver to deliver the infusion fluid from the reservoir into the patient's anatomic space at a substantially constant pressure, a substantially constant force spring mechanism in contact with a pressurizer, and an actuator to gradually load the substantially constant force spring mechanism.

The substantially constant force spring mechanism includes one or two springs, the pressurizer comprises a plunger, and the actuator comprises a lever and a ratcheting mechanism.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate exemplary embodiments of the invention, and together with the description serve to explain the inventive concepts.

FIG. 1A is an illustration of an exemplary embodiment of an infusion system constructed according to the principles of the invention for delivering infusion liquid subcutaneously to a patient.

FIG. 1B is an illustration of another exemplary embodiment of an infusion system constructed according to the principles of the invention for delivering infusion liquid subcutaneously to a patient.

FIG. 2 is a chart of the flow rates, tube sizes, and needle sites used for different drugs to illustrate the need for different variable flow rate controllers for different drugs and needle sites.

FIG. 3 is an illustration of an exemplary embodiment of an infusion system constructed according to the principles of the invention for delivering infusion liquid intravenously to a patient.

FIG. 4A is a perspective view of a variable flow rate controller for use with an infusion system constructed according to the principles of the invention.

FIG. 4B is a cross-sectional view of a variable flow rate controller of FIG. 4A.

FIG. 4C is a cross-sectional view of a variable flow rate controller of FIGS. 4A and 4B showing a decreasing channel and an inlet hole that acts against a slip washer to allow different positions of channels to achieve differing flow rates.

FIG. 5A is a top perspective view of an exemplary embodiment of a butterfly assembly constructed according to the principles of the invention shown in an expanded, open configuration.

FIG. 5B is a top perspective view of an exemplary embodiment of a butterfly assembly with needle constructed according to the principles of the invention.

FIG. 5C is a side sectional perspective view of a butterfly assembly of FIG. 5B.

FIG. 5D is a side sectional view of another exemplary embodiment of a butterfly assembly with needle using a ball-and-pivot joint constructed according to the principles of the invention.

FIG. 5E is an exploded perspective view of a butterfly assembly with needle of FIG. 5B.

FIG. 5F is a front view of an exemplary embodiment of a top portion of a butterfly assembly constructed according to the principles of the invention.

FIG. 5G is a top perspective view an exemplary embodiment of a mating arm of one of the extensions of a butterfly assembly constructed according to the principles of the invention.

FIG. 5H is an exploded perspective view of a butterfly assembly of FIG. 5B with a needle with a needle protector.

FIG. 5I is a schematic front view of an exemplary embodiment of a butterfly assembly constructed according to the principles of the invention in a closed position.

FIG. 5J is a schematic front view of the butterfly assembly of FIG. 5I in a locked position.

FIG. 6A is a perspective view of an exemplary embodiment of a substantially constant pressure syringe pump constructed according to the principles of the invention.

FIG. 6B is a perspective view of an exemplary embodiment of a constant pressure syringe pump of FIG. 6A without a cover.

FIG. 6C is a top sectional view of an exemplary embodiment of a constant pressure syringe pump of FIG. 6A.

FIG. 6D is an exploded view of an exemplary embodiment of a constant pressure syringe pump of FIG. 6A.

FIG. 7 shows a set of calibrated flow dials of a variable flow rate controller of FIG. 4A.

FIG. 8A is a partially exploded view of another exemplary embodiment of a constant pressure syringe pump constructed according to the principles of the invention.

FIG. 8B is a side view of another exemplary embodiment of a constant pressure syringe pump of FIG. 8A.

FIG. 8C is a top perspective view of another exemplary embodiment of a loading mechanism of a constant pressure syringe pump of FIG. 8A.

FIG. 8D shows a top view of another exemplary embodiment of a loading mechanism of a constant pressure syringe pump of FIG. 8C in a loading state.

FIG. 8E shows a top view of another exemplary embodiment of a loading mechanism of a constant pressure syringe pump of FIG. 8C in a neutral load state.

FIG. 8F shows a top view of another exemplary embodiment of a loading mechanism of a constant pressure syringe pump of FIG. 8C in an released loading state.

FIG. 9 shows a simplified mechanism drawing of another exemplary embodiment of a loading mechanism of a constant pressure syringe pump constructed according to the principles of the invention.

### DETAILED DESCRIPTION

In the following description, for the purposes of explanation, numerous specific details are set forth in order to provide a thorough understanding of various exemplary embodiments or implementations of the invention. As used herein “embodiments” and “implementations” are interchangeable words that are non-limiting examples of devices or methods employing one or more of the inventive concepts disclosed herein. It is apparent, however, that various exemplary embodiments may be practiced without these specific details or with one or more equivalent arrangements. In other instances, well-known structures and devices are shown in block diagram form in order to avoid unnecessarily obscuring various exemplary embodiments. Further, various exemplary embodiments may be different, but do not have to be exclusive. For example, specific shapes, configurations, and characteristics of an exemplary embodiment may be used or implemented in another exemplary embodiment without departing from the inventive concepts.

Unless otherwise specified, the illustrated exemplary embodiments are to be understood as providing exemplary features of varying detail of some ways in which the inventive concepts may be implemented in practice. Therefore, unless otherwise specified, the features, components, modules, layers, films, panels, regions, and/or aspects, etc. (hereinafter individually or collectively referred to as “elements”), of the various embodiments may be otherwise combined, separated, interchanged, and/or rearranged without departing from the inventive concepts.

The use of cross-hatching and/or shading in the accompanying drawings is generally provided to clarify boundaries between adjacent elements. As such, neither the presence nor the absence of cross-hatching or shading conveys or indicates any preference or requirement for particular materials, material properties, dimensions, proportions, commonalities between illustrated elements, and/or any other characteristic, attribute, property, etc., of the elements, unless specified. Further, in the accompanying drawings, the size and relative sizes of elements may be exaggerated for clarity and/or descriptive purposes. When an exemplary embodiment may be implemented differently, a specific process order may be performed differently from the described order. For example, two consecutively described processes may be performed substantially at the same time or performed in an order opposite to the described order. Also, like reference numerals denote like elements.

When an element, such as a layer, is referred to as being “on,” “connected to,” or “coupled to” another element or layer, it may be directly on, connected to, or coupled to the other element or layer or intervening elements or layers may be present. When, however, an element or layer is referred to as being “directly on,” “directly connected to,” or “directly coupled to” another element or layer, there are no intervening elements or layers present. To this end, the term “connected” may refer to physical, electrical, and/or fluid connection, with or without intervening elements. Further, the D1-axis, the D2-axis, and the D3-axis are not limited to three axes of a rectangular coordinate system, such as the x, y, and z-axes, and may be interpreted in a broader sense. For example, the D1-axis, the D2-axis, and the D3-axis may be perpendicular to one another, or may represent different directions that are not perpendicular to one another. For the purposes of this disclosure, “at least one of X, Y, and Z” and “at least one selected from the group consisting of X, Y, and Z” may be construed as X only, Y only, Z only, or any combination of two or more of X, Y, and Z, such as, for instance, XYZ, XYY, YZ, and ZZ. As used herein, the term “and/or” includes any and all combinations of one or more of the associated listed items.

Although the terms “first,” “second,” etc. may be used herein to describe various types of elements, these elements should not be limited by these terms. These terms are used to distinguish one element from another element. Thus, a first element discussed below could be termed a second element without departing from the teachings of the disclosure.

Spatially relative terms, such as “beneath,” “below,” “under,” “lower,” “above,” “upper,” “over,” “higher,” “side” (e.g., as in “sidewall”), and the like, may be used herein for descriptive purposes, and, thereby, to describe one elements relationship to another element(s) as illustrated in the drawings. Spatially relative terms are intended to encompass different orientations of an apparatus in use, operation, and/or manufacture in addition to the orientation depicted in the drawings. For example, if the apparatus in the drawings is turned over, elements described as “below” or “beneath” other elements or features would then be oriented “above” the other elements or features. Thus, the exemplary term “below” can encompass both an orientation of above and below. Furthermore, the apparatus may be otherwise oriented (e.g., rotated 90 degrees or at other orientations), and, as such, the spatially relative descriptors used herein interpreted accordingly.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof. It is also noted that, as used herein, the terms “substantially,” “about,” and other similar terms, are used as terms of approximation and not as terms of degree, and, as such, are utilized to account for inherent deviations in measured, calculated, and/or provided values that would be recognized by one of ordinary skill in the art.

Various exemplary embodiments are described herein with reference to sectional and/or exploded illustrations that are schematic illustrations of idealized exemplary embodiments and/or intermediate structures. As such, variations from the shapes of the illustrations as a result, for example, of manufacturing techniques and/or tolerances, are to be expected. Thus, exemplary embodiments disclosed herein should not necessarily be construed as limited to the particular illustrated shapes of regions, but are to include deviations in shapes that result from, for instance, manufacturing. In this manner, regions illustrated in the drawings may be schematic in nature and the shapes of these regions may not reflect actual shapes of regions of a device and, as such, are not necessarily intended to be limiting.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and should not be interpreted in an idealized or overly formal sense, unless expressly so defined herein.

Exemplary embodiments of the invention may be related to certain aspects of the following applications owned by the assignee of this application: PCT Application No. PCT/US2020/051643 entitled “Systems and Methods for Precision Matched Immunoglobulin Infusion,” filed Sep. 18, 2020; PCT Application No. PCT/US2020/051628 entitled “Tissue Saturation Responsive Rapid Automatically Variable Flow Rate Infusion System,” filed Sep. 18, 2020; PCT Application No. PCT/US2020/051592 entitled “Infusion Controller Using Inline Feedback Through Integral Flow Measurement in Tubing,” filed Sep. 18, 2020; and PCT Application No. PCT/US2020/051556 entitled “Infrared Imaging, Measurement, and Analysis of Infusion Sites During Subcutaneous and Intravenous Infusions,” filed Sep. 18, 2020. The disclosures of all these applications are incorporated by reference herein in their entireties,

### Subcutaneous Infusion Example

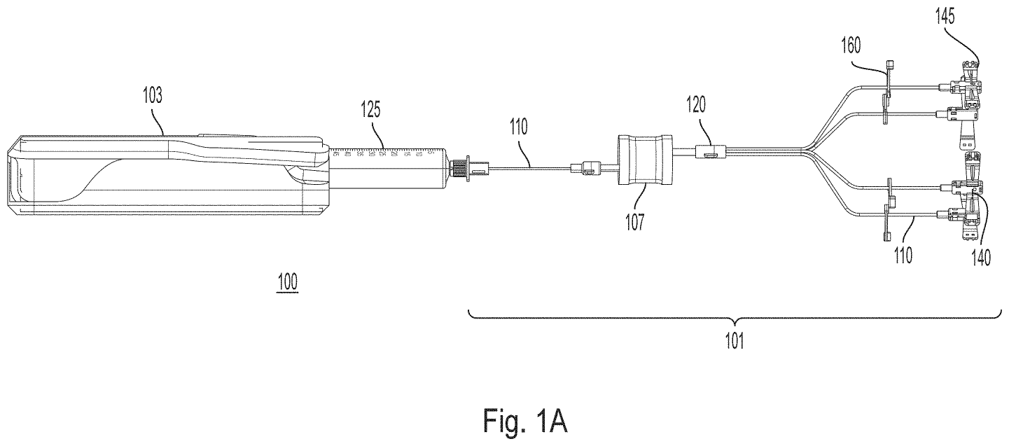

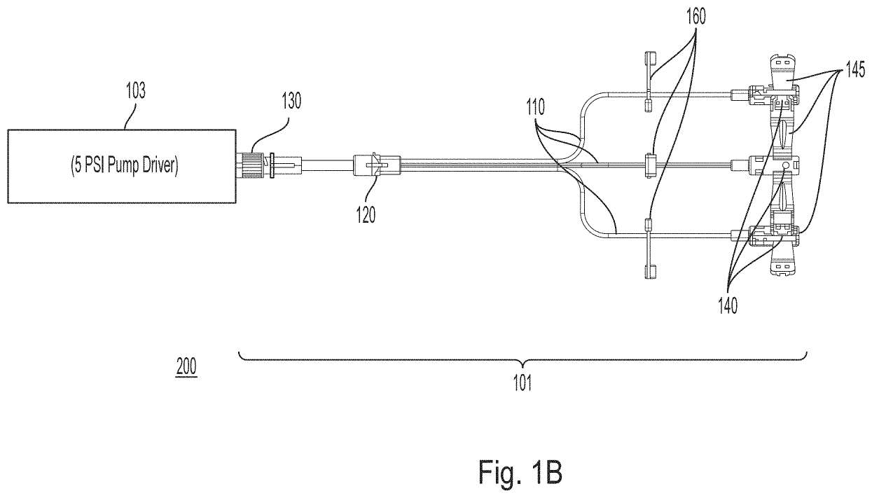

FIG. 1A shows an exemplary embodiment of an infusion system 100 constructed according to the principles of the invention for delivering the infusion liquid subcutaneously to a patient. The infusion system 100 includes an infusion assembly 103 with infusion reservoir 125 and infusion needle set 101.

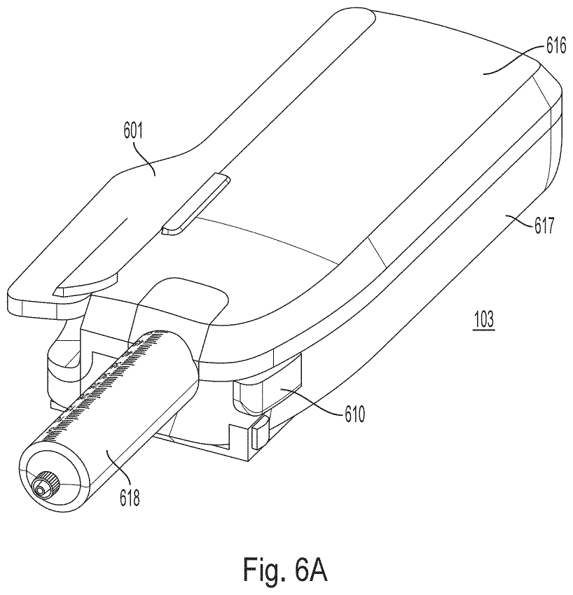

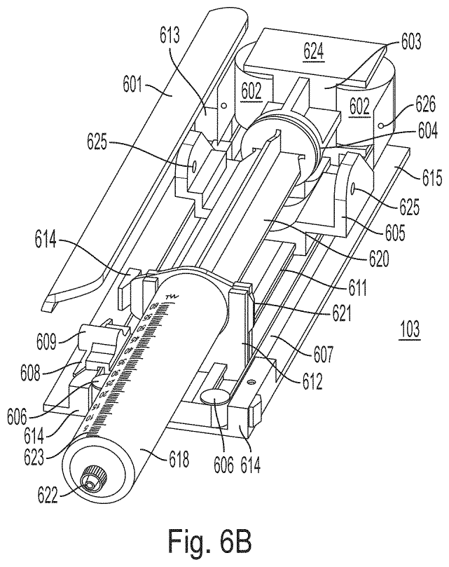

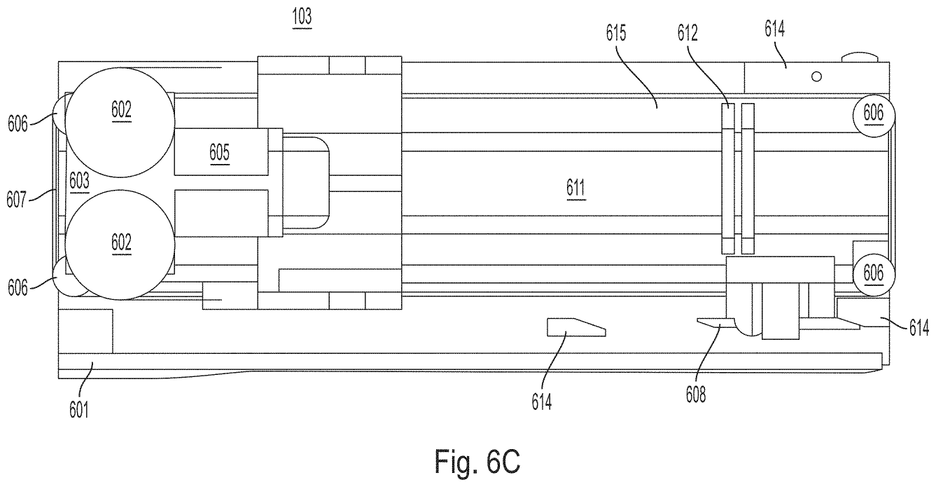

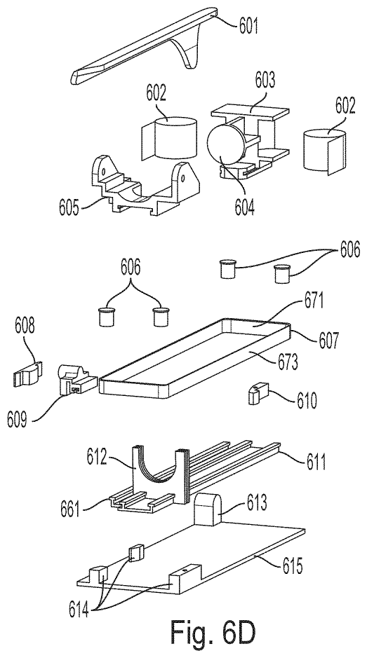

In some exemplary embodiments, as shown in FIG. 1A, the infusion system 100 may be provided to users with the infusion pump assembly 103, which may be a constant pressure syringe driver. The syringe driver assembly 103 is selected based on a need for a particular pressure or amount of liquid over time. The syringe driver (pump) assembly 103 includes a syringe or liquid reservoir 125 and driver (shown in FIG. 6B, including all the parts except those of syringe 618) which drives the syringe to force the fluid in the reservoir into the infusion needle set 101. The infusion system 100 further includes an infusion subcutaneous needle set 101. The infusion subcutaneous needle set 101 includes a variable flow rate controller 107, needle set series tubing 110, manifold 120, tubing clamp/line lock 160, butterfly assembly (connectors/discs) 145, and needles 140.

In some exemplary embodiments, the infusion system 100 is provided to users with only an infusion needle set 101 for use with a patient's own separate infusion driver or pump. In some exemplary embodiments, the infusion driver or pump may be connected with the infusion needle set 101 through any known means, including, e.g., a standard luer disc connector.

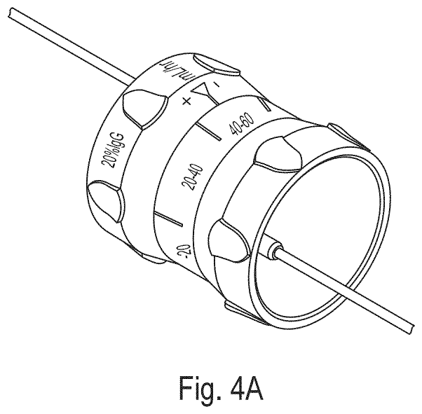

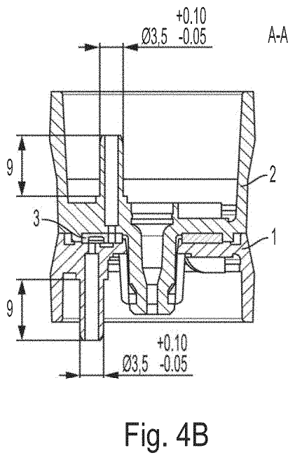

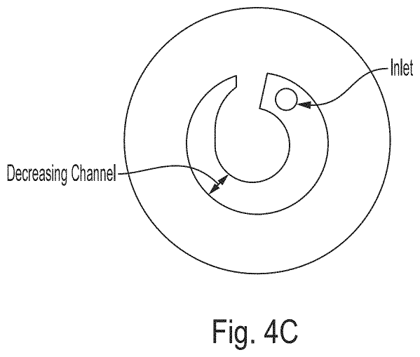

Due to the fluidics of the infusion assembly 103, for subcutaneous administrations, as the number of injection sites is increased, the maximum flow rate per site requires an increased flow rate setting for the controller (shown in FIGS. 4A-C). Thus, the number of needles in the needle set combination requires a different series flow rate regulation. As the number of injection sites increases, the series flow rate equivalent must also increase to regulate and maintain the desired flow rate at the injection sites. In one example, a variable flow rate controller and series precision flow rate tube that is set for the maximum flow rate for use with four-needle sites would create an excessive flow rate beyond the manufacturer's approved drug labeling if used for a single needle site.

In the past, to provide particular flow rates in conventional constant pressure infusion systems, a medical professional would have to change the series tubing 110. That is, the medical professional would have to select different series tubing with a larger diameter/length or a smaller diameter/length. This involves selecting another administration set that may not be immediately available and/or may introduce contamination concerns. Exemplary embodiments of the invention, however, provide advantages as users have access to a variable flow rate controller and can select a number of needle sites to provide or adjust the flow rate of the infusion system. The range of flow rates available with the variable flow rate controller and number of needle sites eliminates the need for stocking specific fixed flow rate administration sets and extension sets and eliminates contamination concerns involved in replacing administration sets or connecting extension sets.

To address this issue, in one exemplary embodiment, a system constructed according to the principles of the invention provides a selection of a different flow rate inlet series tubing to control the maximum flow rate of the system based on the number of needle sites required. Thus, providing a user some additional ways to adjust the flow rate. In one example, as the number of needle sites increases, the flow rate required also increases in order to reach the maximum flow rate at each needle site as stated in the drug manufacturer's package insert. A user of the infusion system may adjust the flow rate controller as well as change the infusion needle set to one allowing a higher flow rate based on the increased number of needle sites.

In the exemplary embodiment of the invention shown in FIG. 1A, the flow rate controller 107 with changed series tubing 110 is used to maximize flow rates of the infusion fluid. In FIG. 1A, the variable flow rate controller 107 has flow rates that are marked in segments of low rates (green or 0-20 ml/hr), medium rates (yellow or 20-40 ml/hr) and high rates (red or 40-60 ml/hr) as shown in FIG. 4A. In this exemplary embodiment, the flow rate controller 107 of FIG. 4A is marked for use only with 20% IgG solutions. In other exemplary embodiments, the variable flow rate controller 107 has flow rates marked at increments of 10 ml/hr (e.g., 10, 20, 30, 40, 50, and 60 ml/hr).

In another exemplary embodiment of the invention, the system may change flow rate controllers 107 for Hizentra® and Cuvitru® or other immunoglobulins for subcutaneous applications based on their infusion rates and viscosity. In other exemplary embodiments, the infusion needle set 101 is selected based on the infused drug, treated health issue, syndrome, or disease, desired flow rate, and number of infusion sites. The flow rate controller 107 and needles 140 are directly connected through the needle set tubing 110 to prevent removal and change of parts of the infusion needle set 101. The needle set tubing 110 extends from the variable flow rate controller 107 to a manifold 120 where the needle set tubing 110 divides into individual needle tubes 110 to needles 140. The needle set tubing 110 includes tubing clamps 160 between sections of tubing, e.g., tubing clamps/line locks 160 on each needle tube and/or the tubing between the variable flow rate controller 107 and manifold 120. The needle set tubing 110 does not include luer connectors due to the infusion needle set 101 being a single piece set for users to select based on situation, e.g., number of infusion sites, infusion fluid viscosity, patient comfort, and infusion fluid maximum infusion flow rate. In some exemplary embodiments, the system is used for a neuromodulation treatment that is subcutaneously administered.

In the exemplary embodiment of the invention shown in FIG. 1A, variable flow rate controller 107 is connected to the series tubing 110 with luer connectors. In some exemplary embodiments, the variable flow rate controller 107 and tubing sets 110 are combined into a single package and connected directly to one another with no middle luer connectors. In other words, the needle set 101 has its own dedicated variable flow rate controller 107.

In some exemplary embodiments, the needles 140 include a butterfly (or disc) assembly 145 for each needle 140 or some variant of butterfly-less and butterfly assembly 145 including needles 140. The infusion needle set 101 generally includes a number of needles 140 between one and eight, however, the number of needles 140 may be greater based on future infusion site allowances and/or changes to needle design. The needles 140 include needles of different bore sizes and lengths, angles of entry, and also are selected for the infusion needle set 101 based on pain control and comfort for a particular patient.

In some exemplary embodiments, the needle(s) 140 of infusion system 100 are inserted into a patient's anatomical space to deliver an infusion fluid. The needle set 101 selected for use is based on a selected infusion fluid and a number of infusion sites. A user or clinician provides a needle set and sets a variable flow rate controller of the needle set to less than or equal to a maximum flow rate of the infusion fluid to be delivered to the patient's anatomical space.

FIG. 1B shows an exemplary embodiment of the infusion system 200 constructed according to the principles of the invention for delivering infusion liquid subcutaneously to a patient. In the exemplary embodiment, the infusion system 200 includes a syringe driver assembly 103 and an infusion needle set 101. The syringe driver assembly 103 may be any infusion pump that is able to generate at least about 5 psi of pressure for the infusion fluid flow and includes an infusion fluid reservoir. In one exemplary embodiment, the syringe driver assembly 103 may be the same infusion assembly 103 of FIG. 1A. The infusion needle set 101 includes a luer connection device 130, tri-connector (manifold) 120, needle tubes 110, slide clamps 160 on each tubing set 110, needles 140, and butterfly assemblies 145 for each needle 140. The syringe driver assembly 103 is connected to the needle set 101, similar to the connection between the infusion assembly 103 and infusion needle set 101 of FIG. 1A, via the luer connection device 130. The infusion system 200 is similar to infusion system 100, except the infusion needle set 101 lacks a flow rate controller.

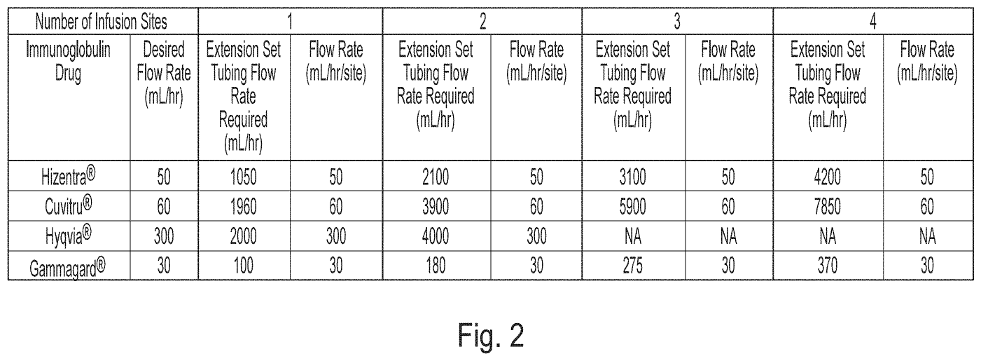

FIG. 2 is a chart of exemplary, calculated subcutaneous flowrates required by each drug, quantity of needle sites, to achieve the flow rates for drugs such as Hizentra®, Cuvitru®, Hyqvia®, or Gammagard® immunoglobulin requiring flow rates between 25 and 300 ml/hr. The infusion system 100 directly provides the same combinations of flow rate selections presented in FIG. 2. For example, specifically for Hizentra® (requiring a flow rate of 50 ml/hr/site), when using a single-needle set, an equivalent flow rate of F1050 is needed. However, if the patient using Hizentra® requires a faster flow rate and/or four-needle sites of infusion, to achieve the same flow rate of 50 ml/hr/site would require an equivalent flow rate of 4200. These custom maximum settings could be either factory set or set by the clinician. The extension set tubing flow rate required flow rate numbers' e.g., 4200, 1050 ml/hr, etc. represent the theoretical water free flow rate required to deliver drug flow rate using a 26 G needle as stated in FIG. 2.

Other drugs of different concentrations and/or viscosities will require different flow rate controllers to limit maximum flow rates dependent on the drug's viscosity. For example, in another exemplary embodiment of the invention, the system 300 may include a particular flow rate controller for Vancomycin or other antibiotics for intravenous applications, which would decrease the required stock of fixed flow rate administration sets by health care providers.

In other exemplary embodiments of the invention, different variable flow rate controllers 107 are required for different situations, dependent on the viscosity of the drug used, which results in changes to the labelling of the flow rate controller for different treatment protocols for neuromodulation versus PIDD, to limit the flow rate to the maximums for each treatment protocol.

### Intravenous Infusion Example

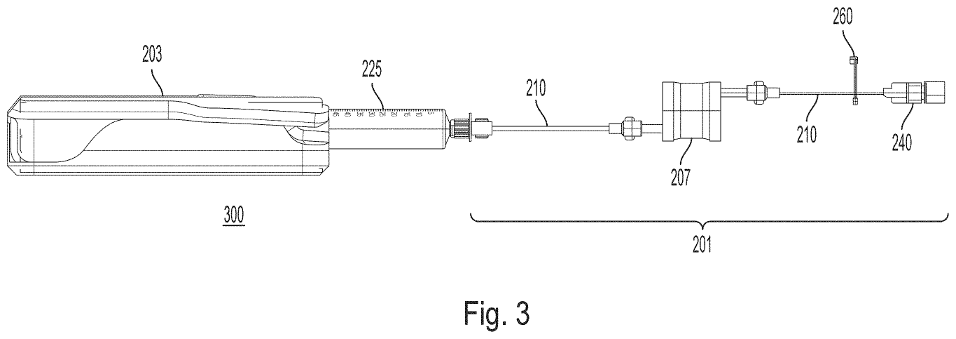

FIG. 3 shows an exemplary embodiment of an infusion system 300 constructed according to the principles of the invention for delivering infusion liquid intravenously to a patient. The infusion system 300 including an infusion intravenous tubing set 201 and infusion driver 203 with infusion reservoir 225. The infusion intravenous set 201 including series tube 210, a variable flow rate controller 207, and a distal luer connector 240 to connect to an IV bag or catheter (not shown separately). The exemplary embodiments of FIG. 3 are similar to those of FIG. 1A above, except as related to the needles and butterfly wings.

### Variable Flow Rate Controller

In FIG. 4A, the variable flow rate controller 107 used with the infusion systems 100 and 300 in some exemplary embodiments of the invention may include custom flow rate controls on the flow rate controllers 107 to set minimum and maximum flow rates or single flow rates. Two inner wheels connected to the main rotational shaft have the ability to set a maximum flow rate and a minimum flow rate. This is accomplished with a series of pin settings (similar to those used to control electric timers), a gear system which disengages from the main drive for setting the flow rate controller, or two settable discs (similar to those used on electric timers on/off controls). In some exemplary embodiments, these controls are lockable using a restricted key design, so that any settings made by the factory or by the clinician cannot be changed by the patient. However, limiting the patient access may be unnecessary because the set range would be, in some exemplary embodiments, safe for patient control.

This flow rate controller is best understood by visualizing the turning shaft of the main controller body is connected to a disc with adjustable slots to impinge upon a fixed shaft on the bottom controller body that can change flow rates in either direction, where one direction further opens/increases the flow rate, and the other direction closes/decreases the flow rate. Further, these slots can be adjusted such that no motion is permitted above or below from a desired flow rate setting, thus turning the variable flow rate controller 107 into a fixed rate controller delivering only a single fixed flow rate.

In particular, as shown in FIGS. 4B and 4C, the variable flow rate controller 107 shows two reciprocal halves of the controller body mounted together on the main shaft with the disc in between such that both end of the slots can be adjusted to any position within the 350-degree rotation limits of the two outside parts of the controller. As a user turns the main controller body, it impinges on the gasket that further impinges on the decreasing channel (shown in FIG. 4C) to limit the flow or increase flow (when rotated in the opposite direction).

Both ends of the slots are adjustable to minimum and maximum values and can be placed so that no interference in the rotation occurs or that the rotation is totally limited to one position or flow rate desired, turning the variable flow rate controller 107 into a single rate fixed system.

In some exemplary embodiments, the variable flow rate controller 107 includes color coded markings for different ranges of flow rates. Thus, more clearly indicating the actual flow rate at the patient through the infusion needle set 101. These indicators may include ranges such as 0-20 ml/hr, 20-40 ml/hr, and 40-60 ml/hr for subcutaneous applications. Further, the indicators may be color coded for green, yellow, and red, respectively to represent low, medium, and high flow rates and potential use danger zones.

In some exemplary embodiments, the variable flow rate controller 107 includes color coded markings for different ranges of flow rates ranging from about 5-about 300 ml/hr for intravenous applications. Further, the indicators may be color coded for green, yellow, and red respectively to represent low, medium, and high flow rates and potential use danger zones.

In some exemplary embodiments, a system 100 includes special packaging that allows infusion providers to adjust the flow rate ranges while maintaining sterility of the infusion needle sets 101. Since the variable flow rate controller 107 is in the same package as the administration needle sets 101 or tubing sets 110, a double pouch arrangement is designed to allow the clinician to adjust the flow rate ranges or single flow rate without jeopardizing the sterility of the needle sets or tubing sets. This unique packaging isolates the needle sets 101 or tubing sets 110 from a separate compartment housing the variable flow rate controller 107, which permits access to the settings.

In some exemplary flow rate controller embodiments, a variable flow rate controller 107 includes different lock-on labelling for specialty flow rate markings. The controllers may include custom flow rate markings for different ranges or for specific drug deliveries. These bands may snap into place either at the factory or by the clinician as desired.

In some exemplary flow rate controller embodiments, a variable flow rate controller 107 includes a keyed locking mechanism, which allows the variable flow rate controller to be delivered either in a fixed flow rate, or in fixed flow range.

In some exemplary embodiments, a variable flow rate controller 107 will be pre-set to the maximum flow rate range of the highest flow rate needed for each combination of needle sets. This results in different settings as more needles are required, since higher flow rates are needed to deliver the liquid to the patient at a set flow rate. This also prevents creating flow rates too fast for a single-needle or a two-needle set.

In some exemplary embodiments, the needle sets use a 26 g needle with 0.036 in+ tubing. In some exemplary embodiments, the connectors have even larger dimensions. In some exemplary embodiments, the tubing includes soft tubing.

In one exemplary embodiment, a variable flow rate controller 107 is set to different ranges but used only for specific treatments and needle sets. For example, for PIDD, one range can be limited to 2400 ml/hr while for a four-needle set for Cuvitru and in another instance, the range of the variable flow rate controller 107 is set at a maximum of 5600 ml/hr with a four-needle set and set to 3200 ml/hr for a two-needle set. In other words, the system would be limited for safety and changeable by the Infusion Provider as needed.

In one exemplary embodiment, a variable flow rate controller includes a channel (FIG. 4C) of variable width and circular length, and by an outside ring rotating around the channel. The flow rate controller can be used to select different channel widths and lengths, which result in different flow rates. By controlling the depth, width, and length of the channel, a wide range of different flow rates can be generated from a single (variable flow rate) controller. The input flow arrives from a series tube on one side of the controller and the output is delivered out the other side of the controller. The variable flow rate controller includes a sliding mating sealing washer and “O” rings to prevent leakage around the channel and rotating shafts.

In some exemplary embodiments for subcutaneous infusion systems, the system packaging includes a complete variable flow rate controller 107 and needle set in one package to provide a single sterilized assembly and luer lock fitting to the pump of the syringe driver. In some exemplary embodiments for intravenous infusion systems, the system packaging includes a complete variable flow rate controller 107 and tubing set in one package, to provide a single sterilized assembly and luer lock fitting to the syringe driver.

FIG. 4B shows an exemplary variable flow rate controller 107 with (1) the interface between the two halves that select the channel location as the one side (2) is rotated into different positions with respect to (3).

As outlined above, FIG. 4C shows a cross-sectional view of the variable flow rate controller 107 with the disc and main controller body not showing. The cross-sectional view shows a channel which decreases in width to limit or increase the fluid flow. The decreasing channel and an inlet hole that acts against a slip washer to allow different positions along the decreasing channel to achieve different flow rates. FIG. 4C shows a decreasing channel (width) in one half the controller, which is selected by rotating the controller halves to select different points in the channel path. The channel varies by width and depth and is then selectable by length to obtain any desired flow rate setting.

Infusion systems and methods in accordance with some exemplary embodiments of the invention accurately and reproducibly deliver an infusion fluid to a patient at a desired anatomical location by allowing for direct control of the infusion system pressure. Patients and clinicians can determine the infusion system flow rate and deliver a volume of an infusion liquid at a speed that does not cause discomfort. Patients and clinicians and other users can match the infusion liquid and needle sites (for subcutaneous applications) and variable flow rate controller settings to increase the probability of safe treatment using the infusion system. A patient or clinician can set these system variables and immediately determine which treatment configuration is best for the treatment type.

### Butterfly Assembly

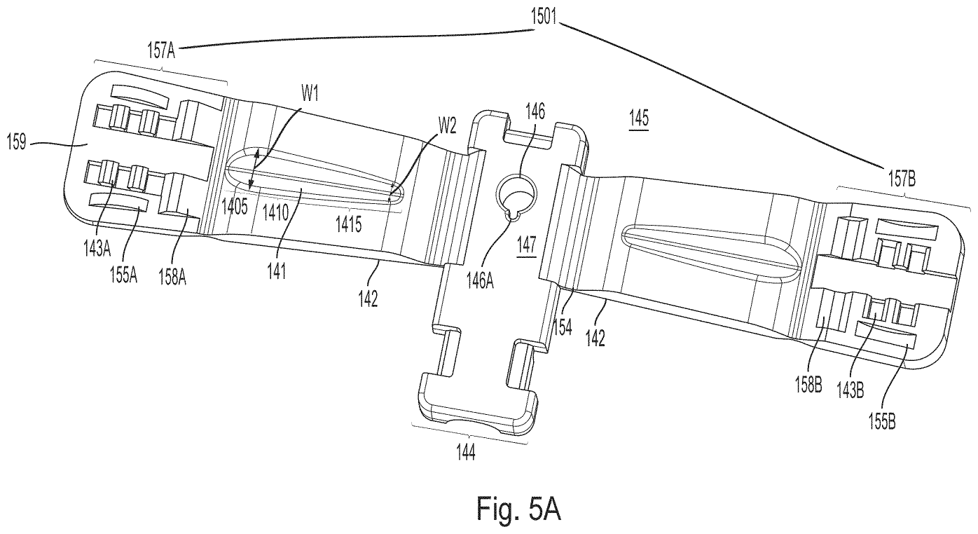

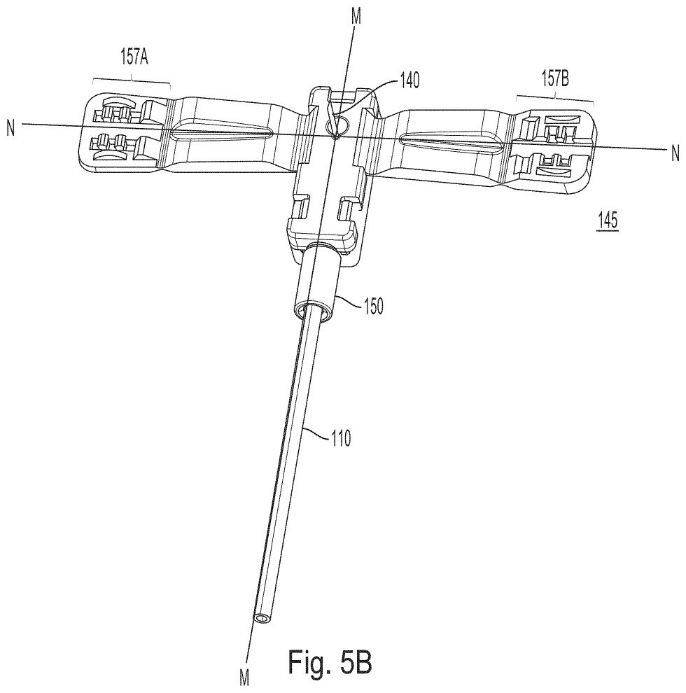

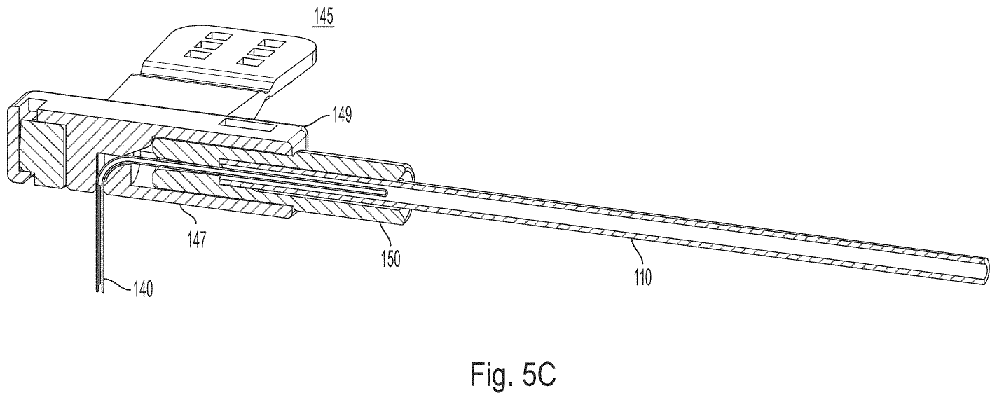

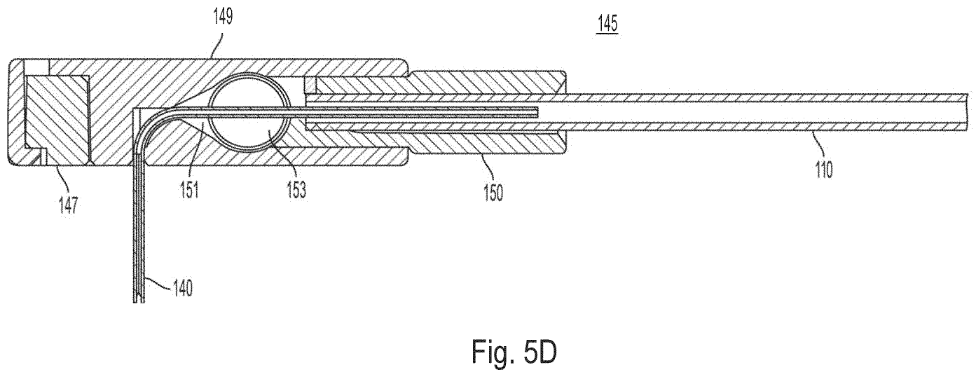

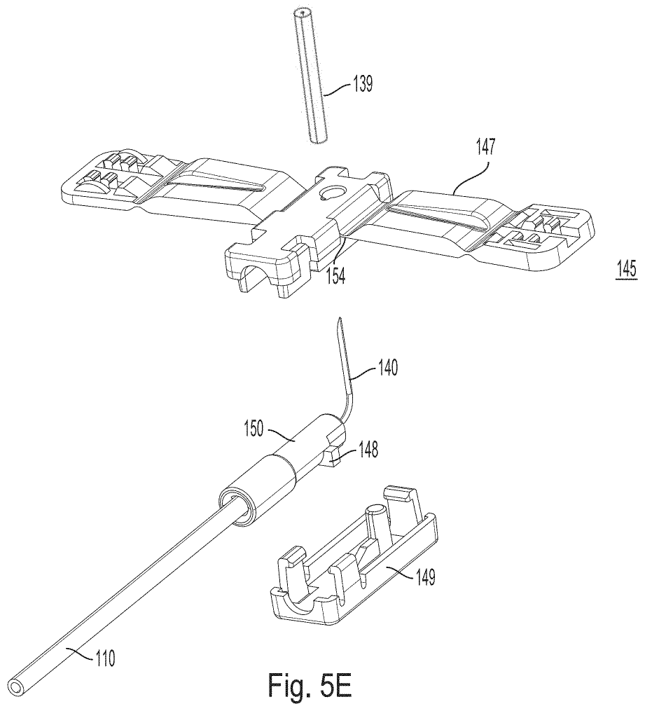

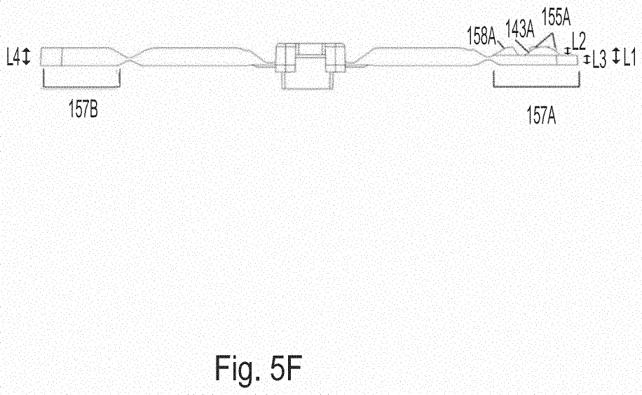

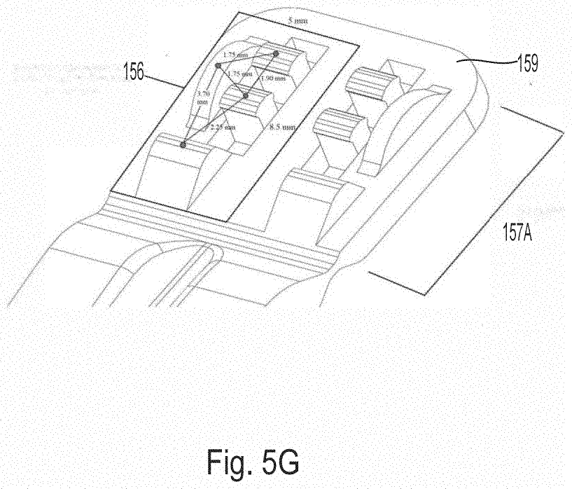

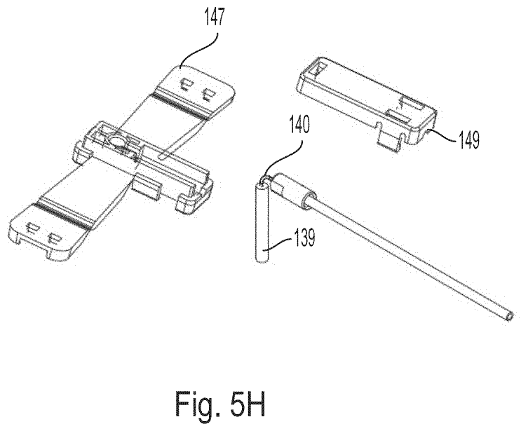

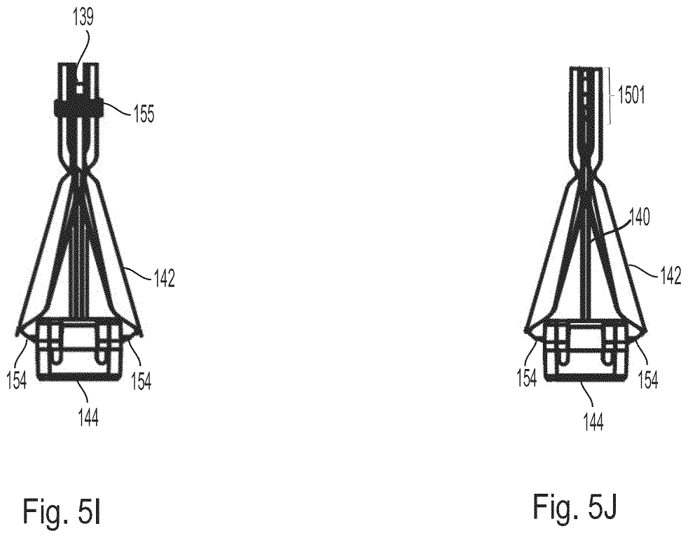

FIG. 5A shows a top perspective view of an exemplary embodiment of a butterfly assembly constructed according to the principles of the invention in an expanded, open position. The exemplary embodiment of the butterfly assembly 145 includes a body, which may be in the form of a hub 144 and a pair of extensions hinged to the body which may be in the form of butterfly wings 142. The butterfly assembly 145 may be made from any soft plastic, for example, polypropylene (PP), polyvinyl chloride (PVC), polycarbonate (PC), and thermoplastic polyurethane (TPU). The wings 142 are movable between the expanded, open position shown in FIG. 5B and the closed, compact and locked positions shown in FIGS. 5I and 5J, respectively. The butterfly assembly is shipped from the manufacturer to a user in the closed, compact and unlocked position in which in which the wings are held together by a rubber band 155 or similar closure, as shown in FIG. 5I. As disclosed in more detail below, after use the wings may be locked together in the locked position by mating locking features on the wings to surround the needle and prevent a needle stick injury, as shown in FIG. 5J.

The hub 144 includes a needle 140 (shown in FIGS. 5B-5D) which includes a needle seat or connector 150 that holds the needle in place in a notch 146A in the needle access opening 146. The needle 140 connects to the rest of the needle set 101 via the needle (series) tubing 110. Thus, the notch 146A receives the needle, which is retained in a position substantially orthogonal (i.e., out of the page of FIG. 5) to the length of the housing 147 by the connector 150, which is attached to the bottom of the hub 144.

FIG. 5B shows a top perspective view of an exemplary embodiment of a butterfly assembly with needle constructed according to the principles of the invention. The needle 140 and needle seat 150 connected to the butterfly assembly 145. This may be accomplished by the following steps with reference to FIGS. 5E and 5H. The substantially straight needle 140 is glued into the inner diameter of one end of the needle seat 150. The needle protector 139 is then placed on the straight needle. The needle 140 is then bent to a 90 degree angle. The needle tubing 110 is glued into the inner diameter at the other end of the needle seat 150. The subassembly (110, 140, 148, 150) is then threaded into the needle access opening 146. The needle seat ribs 148 locks into rib recesses in the butterfly housing 147/145 and forces the needle to move down into the notch 146A and prevents the needle from moving in the direction of the long axis of the hub and the long axis of the wings.

FIG. 5C shows a side sectional perspective view of a butterfly assembly constructed according to the principles of the invention. The needle seat 150 is placed between a butterfly cover 149 and butterfly housing 147 to hold the needle in place when placed in the butterfly assembly 145. As shown also in FIGS. 5E and 5H, the cavity between the butterfly cover 149 and butterfly housing 147 further includes a needle holding and guiding path and space to trap the needle seat 150. The needle seat 150 may further include spacers 148 that mate with the butterfly housing 147 and/or butterfly cover 149 to hold the needle 140 in a position.

In the manufacturing process the needle is secured in the needle access opening 146 and a needle protector 139 is placed around the needle to maintain needle sharpness and avoid needle tip damage during the manufacturing process. In use, the needle protector 139 is removed and a drug may be administered by pressing the butterfly assembly 145 to a patient's skin where the needle 140 is inserted for subcutaneous administration. The butterfly assembly 145 includes special features discussed below to ensure the safety and comfort of the patient, including lack of any memory or significant biasing forces tending to force the extensions into either position, and increased contact area on the interior surfaces of the wings presented to the patient's skin to diffuse the contact force over a greater area and reduce pain or discomfort.

The butterfly assembly 145 is connected in series with and in the same direction as the length of the series tubing 110. As noted above, the butterfly assembly 145 houses the needle 140 such that the needle protrudes both orthogonally to the longitudinal axis of the butterfly assembly and to the series needle tubing. In one exemplary implementation, the needle 140 (as shown in FIG. 5B) may be bent to achieve this orthogonality. Furthermore, the butterfly housing 147 (FIG. 5A) have symmetrically positioned butterfly wings 142 extending outward from the hub 144. The butterfly wings 142 are used as a needle insertion/removal handling feature and conform to the patient's skin without causing irritation or discomfort. The butterfly wings 142 include a mating arm 157A and a recessed arm 157B, both of which include locking structures that may be in the form of locking mechanism having corresponding closing features including tab 143A and slot 143B connections which mate together to guide and lock the arms 157A, 157B together in a locked position. As shown in FIG. 5J, a schematic front view of the butterfly assembly of FIG. 5I in a locked position. The butterfly wings 142 protect the needle after use to eliminate potential harm (e.g., needle-stick injuries) when in the locked position. To protect the needle 140 after use, the butterfly wings 142 may be closed and locked together with the fingers of a single hand by pressing the mating arm 157A and recessed arm 157B together such that the locking structures engage as described below.

In FIG. 5A, the locking mechanism 1501 is used to keep the wings 142 in a locked position and includes the corresponding closing features of the mating arm 157A and recessed arm 157B. The locking mechanism 1501 may include a double closure (e.g., protrusions 143A), in which both butterfly wings 142 have a closure configured to mate with the opposing wing. In this exemplary embodiment, the closure includes one or more protrusions 143A and one or more corresponding recesses 143B to receive the protrusions 143A. The locking mechanism 1501 also includes one or more surfacing mechanisms 158A, and one or more guiding mechanisms 155A which include corresponding recessed guiding recesses 15513 (corresponding to the guiding mechanisms 155A) and surfacing recesses 158B (corresponding to the surfacing mechanisms 158A). The locking mechanism 1501 is interior to a surface of the wings 142 to prevent perception by a patient of an edge. Protrusions on edges of the wings 142 would result in discomfort to the patient. The protrusions 143A and recesses 143B mate together to hold the butterfly assembly 145 in a locked position. In some exemplary embodiments, the protrusions 143A and corresponding recesses 143B include tabs and slots, respectively. In some exemplary embodiments, the guiding mechanisms 155A include curve(s) that guide the butterfly wings 142 into a locked position so that, even if misaligned, the butterfly wings 142 will be aligned by the time the wings 142 are in a locked position. In one exemplary embodiment, the guiding mechanisms 155A may include a curved protrusion with a minor circular arc and a segment length along the wing longitudinal axis N-N (see FIG. 5B) that is about 3.5 mm and includes a midpoint height of about 0.85 mm from interior surface 159. In one embodiment, the segment length is between about 2 mm and 6 mm. In some exemplary embodiments, the segment length provides enough length to guide the protrusions 143A and recesses 143B together by being parallel to any and all of the protrusions 143A and recesses 143B that are on the wings 142. In one exemplary embodiment, the guiding mechanisms 155A are in positions mirrored across axis N-N (see FIG. 5B). In some embodiments, the guiding mechanisms may include any number of shapes that are configured to guide the butterfly wings into an aligned locked position.

When the butterfly wings 142 are closing, users will observe sensory feedback, which may be in the form of a tactile and/or audible feedback, including a snap or click indicating to users that the butterfly wings 142 are closed, and the needle tip is protected and shielded from the user (after use of the needle set). Furthermore, the raised, smooth surface topography of the butterfly wings 142 and its locking mechanism avoid the use of any guiding or locking mechanisms=at the periphery of the wing and increases the surface area that contacts the patient during use to create a diffuse patient contact surface that reduces discomfort and pain when placed on the skin.

The butterfly wings 142 can also include grooves designated to guide and maintain the needles' orthogonal (90°) orientation within the needle protector 139 such that the needle is straight and undamaged when received by the user. As shown in FIG. 5A, in one embodiment, the guiding channel 141 is a tapered hemispherical channel along each wing 142 that form a hollow ellipsoid when the wings 142 are folded upon each other about central axis M-M as shown in FIG. 5B. The guiding channel 141 includes three defining sections: the calyx 1405, the cheek 1410, and neck 1415. The calyx 1405 includes the widest portion of the guiding channel 141. The cheek 1410 includes the graduated tapering portion of the guiding channel 141. The neck 1415 includes the narrowest portion of the guiding channel 141. The channel 141, of FIG. 5B, captures the protected needle when both wings 142 are in a closed but unlocked position shown in FIG. 5I. This ensures that the needle is retained in a substantially 90° state before use and penetrates to the correct skin tissue depth, thereby avoiding the associated discomfort and pain from improper needle penetration as a result of an angled needle. For example, if the needle is bent at an angle other than 90° and is inserted into the patient's skin, the needle may no longer be at the correct penetration depth to deliver the drug properly to the patient's subcutaneous space. Also, if the needle is bent at an angle other than 90 degrees, the needle may penetrate the skin incorrectly and unnecessarily damage tissue, or may cause pain and/or discomfort to the patient.

As shown in FIG. 5A, the butterfly wings 142 guiding channel 141 diameter increases from about 1.0 to 2.5 mm laterally from the butterfly housing 147. In this exemplary embodiment, the guiding channel 141 is used in conjunction with a needle protector 139 shown in FIGS. 5E, 5F, and 5H. The body of the needle protector 139 takes the form of a about 2.0 mm diameter cylinder that slides over the needle 140 to protect the needle tip. The needle protector 139 and encased needle 140 is trapped, and movement is prevented entirely by the narrower section of channel 141 (neck 1415 to cheek 1410), which is tapered such as from about 1.0 to 2.0 mm in width. The needle protector 139 is trapped and guided into the wider section of the guiding channel 141 (cheek 1410 to calyx 1405), which is tapered such as from about 2.0 to 3.0 mm in width when the butterfly assembly 145 is closing or in a closed position and provides an encasement by the tapered guiding channel 141. The taper provides space to capture a misaligned needle protector 139 and direct the protector, and therefore needle in toward the center of the guiding channel 141, and thus, maintains the substantially 90° bent angle of the needle. As shown in FIG. 5I, a schematic front view of an exemplary embodiment of a butterfly assembly constructed according to the principles of the invention in a closed position. A rubber band 155 is placed around the butterfly wings and encased needle protector 139 and needle 140 causing the butterfly wings to apply a compressive force around the needle protector 139. Given this, a needle protector and thus its encased needle is limited in movement and can be realigned if misaligned during the manufacturing process or shipping because of the butterfly wings' needle guiding channel.

In one embodiment, the guiding channel 141 is not a true hemisphere, rather the guiding channel 141 includes tapered arcs that transition from the surface of the butterfly wing 142 to allow the needle protector 139 to be guided into the channel 141. Some examples of needles 140 may include 28 to 24 gauge needles (about 0.37 mm to 0.58 mm outer diameter respectively). As such, an associated needle protector 139 would have an outer diameter of at most about 2.00 mm wider than the gauge of the needle, e.g., 28 and 24 gauge needles have needle protectors 139 with outer diameters of 2.37 mm and 2.58 mm. In some exemplary embodiments, the needle protector 139 outer diameter would remain the same, however the inner diameter would change for each needle gauge. For example, the needle protector 139 may always be 2.00 mm in outer diameter, but whose inner diameter may be just large enough to smoothly fit each gauge of needle.

To accommodate these needles 140 and associated needle protector(s) 139 the tapered guiding channel 141 may include W1 widths, for example, between about 2.37 mm and 3.00 mm for 28 gauge needles and about 2.58 mm and 3.00 mm for 24 gauge needles. In other words, the W1 width is at least wider than the diameter of the needle protector 139. Additionally, the tapered guiding channel 141 may include W2 widths, for example, about 1.58 mm for 24 gauge needles with a about 2.58 mm needle protector 139. In other words, width W2 is smaller than the diameter of the needle protector 139 by at most about 1.0 mm. For example, for a about 2.0 mm diameter needle protector 139 the tapered guide channel 141 has a width W1 between about 2.0 mm and 3.0 mm and width W2 of about 1 mm. In other words, as long as, the maximum outer diameter of the needle protector 139 can be restrained by the narrower width section W2 of the neck 1415 of the guiding channel 141, then the guiding channel 141 will be capable of maintaining the 90° angle of the needle. For example, a needle protector 139 with an outer diameter of 1.1 mm and guiding channel 141 with width W2 of 1.0 mm would still work since part of the outer circumference of the needle protector 139 would roll into the guiding channel 141.

In other exemplary embodiments, such as illustrated in FIG. 5D a side sectional view of another exemplary embodiment of a butterfly assembly with needle using a ball-and-pivot joint constructed according to the principles of the invention. The butterfly assembly 145 in combination with needle 140 can include a ball-and-pivot or floating ball mechanism such that when inside of the butterfly housing 147, the needle 140 may rotate (e.g., five degrees) in any direction at the pivot socket (point) 151. The ball-and-pivot mechanism includes a ball 153 which mates with the needle 140 to hold a portion of the needle 140 in position while allowing rotation, within the pivot socket 151, of the interfaced ball 153 and needle 140. In this fashion, slight motion of the butterfly assembly does not transmit to the needle and does not cause the needle to move within a patient's tissue. As a result, needles in accordance with some exemplary embodiments of the invention eliminate motion forces transmitted through the needle during an infusion, which can otherwise damage tissue and cause pain and inflammation. The pivoting needle feature eliminates tissue damage and pain by rotating the needle at the pivot and within the butterfly housing in response to forces placed on the butterfly assembly.

In one exemplary embodiment, as shown in FIGS. 5A, 5B, 5E, and 5F, the butterfly wings 142 are flexible and have “no memory,” i.e., viscoelastic properties of the hinge 154 prevent permanent deformation. In other words, the hinge 154 between the wing 142 and hub 144 has viscoelastic characteristics that result in the hinge remaining in a neutral and un-compressed state when in the substantially flat expanded position. In other words, the hinge is designed to allow the wings to be movable between a closed, compact position and an expanded, open position without any significant biasing force tending to force the wings into either position. This is advantageous since the butterfly wings 142 are designed to lay substantially flat on the patient's skin and any elastic characteristics (internal memory or biasing forces) may result in pressing against a patient's skin. For example, elastic properties in other butterfly wings may cause the wings to permanently deform in a way that forces the wings together towards the needle, thus resulting in the wings pressing against the patient's skin in an uncomfortable way. This pressing can cause the inserted needle to recede from the penetrated subcutaneous space. As a result, the needle may no longer be at the correct penetration depth to deliver the drug properly to the patient's subcutaneous space. Additionally, simply pressing against the patient's skin may cause pain, discomfort, or irritation to the skin (such as skin discoloration). In one embodiment, the butterfly wing 142 with the “no memory” hinge 154 may be less than about 0.3 mm in thickness, preferably less than or equal to about 0.2 mm in thickness, and will stay in the substantially flat position when opened within a range of about 20 degrees, i.e., about 10 degrees above and about 10 degrees below the neutral horizontal plane. Any thicker, such as greater than about 0.3 mm, will cause the butterfly wing 142 hinge 154 to move, i.e., permanently deform, from the neutral state. In one exemplary embodiment, the hinge 154 may include about a 0.2 mm thick rib running orthogonal to the length of the hinge 154. The ribs may further reduce stresses in the hinge 154 and thus reduce opposite effects on the wings 142 in a closed or locked position.

In one exemplary embodiment, the butterfly wings 142 further allow single-handed use with hinges 154 and locking mechanism 1501, which may be closed with small closing forces such as less than or equal to about 3 lbs and opened by requiring a relatively high force of greater than or equal to about 6 lbs to open the locking mechanism 1501, which is designed to prevent the user from opening the butterfly after the needle was used for an infusion and thereby avoid needle stick injuries. The locking mechanism 1501 of the butterfly wings 142 relies upon protrusions 143A and recesses 143B which snap together. In one exemplary embodiment, the number of protrusions is two or less to a side to prevent misaligned closure since more protrusions and recess combinations would allow accidental protrusion insertion to the wrong recess. In one exemplary embodiment, the snap locking mechanism 1501 provides a tactile and/or auditory feedback when moved to a locked position to make sure the user knows the butterfly wings 142 are safely closed.

FIGS. 5F and 5G show a front view of an exemplary embodiment of a butterfly wing 142 and a top perspective view of a mating arm 157A of a butterfly wing 142, respectively, constructed according to the principles of the invention. Immunoglobulin patients have skin that is sensitive and prone to irritation. As a result, the butterfly wings' 142 contact area 156 with the skin is designed to be smooth to the touch of a patient. The butterfly wing 142 mating arm 157A includes locking protrusions 143A, surfacing mechanisms 158A (or other protrusions), and guiding mechanisms 155A which are designed to provide an adequate surface area such that the butterfly wings 142 and their mating arm 157A and recessed arm 157B are substantially smooth to the patient's skin such that the protruding features are imperceptible or nearly imperceptible to the patient due to smooth or rounded edges and a relatively large contact surface area that is spread out over the surface area with protrusions at a set distance from one another. These features diffuse pressure points to eliminates discomfort, irritation, or pain to the patient's already sensitive skin.

As shown in FIG. 5F, the butterfly wing 142 mating arm 157A includes features such as the surfacing mechanisms 158A (see FIG. 5A) and guiding mechanisms 155A (see FIG. 5A) that are raised from a mating arm 157A interior surface 159 (see FIG. 5A) to create a more even topographical surface and prevent the patient from feeling the protrusions (including the surfacing mechanisms 158A, guiding mechanisms 155A, and locking protrusions 143A). The butterfly wing 142 recessed arm 157B includes recesses 158B and 155B to receive the surfacing mechanisms 158A and guiding mechanisms 155A respectively. This makes the recessed arm 157B smooth and minimizes skin irritation. On the opposing mating arm 157A of butterfly wing 142, there are ramps 158A, curves 155A and tabs 143A that protrude from the interior surface 159 of the arm 157A to mate with the opposing butterfly wing's 142 recessed arm 157B recesses 158B, 155B when closed. These features protrude at or near the same height from the interior surface 159 of the mating arm 157A and thus prevent one of the features from protruding more than another, which may introduce irritation due to the difference in the height of the features becoming perceptible to the patient.

Further, as shown in FIG. 5G, in one exemplary embodiment, the features are clustered within an interior surface 159 of the mating arm 157A. Combined protrusion surfaces (the area shown in FIG. 5G) can have a about 42.5 mm² surface area and can be positioned about 5.0 mm or less from nearby features on the same half of the mating arm 157A measured from the maximum height point of the locking protrusions 143A, surfacing mechanisms 158A, and guiding mechanisms 155A. For example, as shown in FIG. 5G, the top center point of each protrusion (each ramp, curve, and locking feature) is spaced by a distance between about 1.75 mm and about 3.70 mm and each edge is rounded to prevent tactile perception of topographical spaces between the protrusions to a patient. In some exemplary embodiments, the surfacing mechanisms 158A include ramps and other protrusions with smooth protruding surfaces in contact with the patient's skin to spread out contact along the interior surface 159 of the mating arm 157A. The height L2 of the protruding features should be designed such that, when considering the thickness L1 (the distance from the bottom to the top of the butterfly wing 142) of the mating arm 157A, i.e., the sum of the height L2 of the protruding features (e.g., ramps 158A, curves 155A, tabs 143A, and other smooth protrusions) and the thickness L3 of the interior surface 159 of the mating arm 157A, is the same or near the same as the thickness L4 of the recessed arm 157B. In one exemplary embodiment, the mating arm 157A surfaces in contact with the patient's skin are smooth to the touch with generally curved or ramped surfaces to make the mating arm 157A contact surface imperceptible to the patient.

In one exemplary embodiment, as shown in FIG. 5F, the thickness L4 of the recessed arm 157B is about 1.80 mm. As such, the summed height L1 of the protruding features (about 0.95 mm) L2 and the interior surface 159 thickness L3 of mating arm 157A (about 0.85 mm) is about 1.80 mm. Both the mating arm 157A and recessed arm 157B thicknesses, L1 and L4 respectively, should not exceed an overall thickness of about 3 mm. For the mating arm 157A, the height L2 of the protruding features should be around 50% of the thickness of the summed thickness L1. A height L2 of less than 50% of the summed thickness L1 may result in poor latching forces when the butterfly wings 142 are closed.

When using an administrative set with a butterfly assembly according to the principles of the invention, drugs are introduced into the administration set from a fluid reservoir, such as a syringe, via a mechanism, usually a syringe driver or infusion pump. The tubing set is filled up to the needle with the drug or medication, thus purging the set of air. When using medications other than immunoglobulins, purging through the needle may be acceptable.

After swabbing the skin with alcohol and waiting several minutes, a needle is inserted into the patient. Several needles may be used/inserted in the case of multi-needle administration sets. The needle is housed by a butterfly assembly that includes a pair of butterfly wings that are pressed flat onto the surface of the patient's skin during the infusion. As noted above, the butterfly assembly may be packaged with the needle in the closed, compact position, but not locked using a band or tie around the ends of the wings), before use. Before use, the user opens the butterfly assembly by spreading the wings apart to expand the butterfly assembly and removes the needle protector from the needle. The needle of the butterfly assembly is then ready for insertion into the patient's anatomic space. An adhesive dressing may be used over the butterfly assembly and needle to minimize butterfly assembly and needle movement.

The syringe driver or infusion pump or other mechanism used to deliver the drug or medication from the fluid reservoir is activated, and the drug is administered to the subcutaneous space under the patient's skin via the administration set. After the infusion is completed, the needle(s) are removed from the patient with a single-handed technique, and the wings of the butterfly assembly are securely pressed together to close around the needle to prevent needle stick injuries. The butterfly assembly encases the needle, after use, in the butterfly wings and shields the patient from the tip of the needle to prevent needle stick injuries. A guiding mechanism may guide the wings to prevent misalignment of the wings in the locked position. The needles can then safely be discarded in an appropriate sharps container.

### Infusion Driver

FIG. 6A shows a perspective view of an exemplary embodiment of a substantially constant pressure syringe pump constructed according to the principles of the invention. In one exemplary embodiment, a substantially constant pressure mechanism may be in the form of a substantially constant pressure syringe pump assembly 103. The pump assembly 103 includes a syringe 618 acting as a reservoir and including a pressurizing mechanism to dispense infusion fluid from the syringe 618, which may be in the form of a syringe plunger 620 as shown in FIG. 6B). The plunger 620 is one example of a pressurizer to drive the infusion fluid from the reservoir (e.g., syringe 618) into a patient's body. The pump assembly 103 also acts as a housing for the syringe 618. The body of the housing including a main body portion 617 and cover 616. Further, the pump assembly 103 includes an open button 610 to remove the cover 616 from the body 617, and a lever 601 to actuate the pump assembly 103 and dispense infusion fluid from the syringe 618 at a substantially constant pressure.

FIG. 6B shows a perspective view of an exemplary embodiment of a constant pressure syringe pump according to the principles of the invention without a cover. The constant pressure syringe pump assembly 103 includes a mechanism for mating with the syringe plunger 620 to accurately actuate the syringe plunger 620.

As shown in FIGS. 6A-6D, an exemplary embodiment of the constant pressure syringe pump assembly 103, when not in use or when the lever 601 and cover 616 are closed against the body casing 617 of the pump assembly 103, the pump assembly 103 is in its most compact form. To operate the pump assembly 103 in this condition, a user must first engage a cover opening button 610 that allows the lever 601 and cover 616 to open to some degree.

In an exemplary embodiment of the constant pressure syringe pump assembly 103, the pump assembly 103 actuating mechanism is a lever 601. The lever 601 is attached to the lever attachment point 613 that is fixed to one corner of the base plate 615. The lever attachment point 613 protrudes from the base plate 615 such that the attached lever 601 can rotate around the lever attachment point 613. In some exemplary embodiments, the lever 601 is at a length such that 4 strokes at approximately 3.5 pounds of force per stroke is needed to fully load the pump assembly 103 actuating mechanism. In some exemplary embodiments, the cover 616 may also be attached at the lever attachment point 613 and rotate to some degree. Further, a mechanism (e.g., a spring), can be used to aid in opening the lever 601 and cover 616, such that when the pump assembly 103 is in a “not in use” state, the spring is compressed between two structures of the pump assembly 103 such as the cover 616 and base plate 615. When the cover opening button 610 is pressed, the lever 601 and cover 616 are no longer bound to the body casing 617, and the compressed spring can release stored energy and return to its natural position by pushing the cover 616 away from the base plate 615. In other exemplary embodiments, other actuating mechanisms such as buttons or electrically operated motors may be used in place of lever 601.

In an exemplary embodiment of a constant pressure syringe pump assembly 103, once opened, a user may load a pump-specific syringe 618 filled with medication that is unique to the patient's treatment needs. The syringe 618 is connected to an administration set (i.e., a subcutaneous needle set 101 or an intravenous infusion set 201) specific to user treatment needs. The syringe 618 is fitted such that the syringe flange sits securely and is aligned within the syringe flange receptor 612 such that the extended syringe plunger 620 can be received by the syringe plunger receptor 604, which is connected to a substantially constant force spring mechanism that may be in the form of the negator carriage mechanism 603. The syringe plunger receptor 604 is a protruding extension of negator carriage 603 and does not interfere with any other attached component of the pump assembly 103. The syringe plunger receptor 604 is in substantially constant contact with the substantially constant force spring mechanism to provide a substantially constant pressure when in use. The syringe flange receptor 612 is fixed to base plate 615 such that a fully extended syringe plunger 620 of the pump-specific syringe 618 can fit between the syringe flange receptor 612 and the syringe plunger receptor 604. In some exemplary embodiments, the negator carriage 603 may be manually moved back away from the syringe flange receptor 612 such that the syringe 618 may fit within the pump assembly 103.

In an exemplary embodiment, when the pump assembly 103 is not in use, the negator carriage 603 may freely move, within the allowable physical limits, in the direction of the compact (triple) track rail 611. The contact between the negator carriage 603 and the compact (triple) track rail 611 is a low-friction material to enable gliding. Low-friction gliding can be achieved in several manners including the use of ball bearing track contacts or other methods.

In the illustrated embodiment, the negator carriage 603 symmetrically houses two specified force negators 602 (also called constant force springs). The negators 602 are fitted onto posts of negator carriage 603 using low-friction bearings such that negators 602 do not exhibit drag or high frictional forces on the negator carriage 603 when active. The negators 602 are positioned such that they are mirrored about the midline long axis of the negator carriage 603. The negators 602 are placed such that their inner diameters are positioned substantially in parallel to the base plate 615. The negators are further positioned such that when unspooled, the internal surface of both negators 602 will face towards the compact (triple) track rail 611. Further, the negators 602 are symmetrically positioned onto the negator carriage 603 (and thus also a longitudinal axis of the fluid reservoir) such that when active, the negators 602 do not exhibit unnecessary torsional forces. In an exemplary embodiment, the negators 602 deliver about 7-10 pounds (lbs.) of force such that the output force is around 13.5 psi of pressure.

In an exemplary embodiment, the negators 602 are secured onto negator carriage 603 with a carriage covering plate. The negators 602 that are attached to the negator carriage 603 are symmetrically positioned onto the compact (triple) track rail 611 such that the negators 602 unspooling direction points in the direction of the compact (triple) track rail 611.

In an exemplary embodiment, between the syringe plunger receptor 604 and the syringe flange receptor 612 is a negator loading carriage 605 that is symmetrically positioned and connected to the compact (triple) track rail 611 similarly to the negator carriage 603. The height of the negator loading carriage 605 is positioned such that it does not interfere with the syringe plunger 620. The negator loading carriage 605 provides two symmetric holes that are specifically placed such that the attachment holes of each negator 602 aligns with the holes of the negator loading carriage 605 such that when connected to the holes of the negator loading carriage 605 and then unspooled, each negator 602 is parallel to the compact (triple) track rail 611. Further, the height of the holes of the negator loading carriage 605 and the height of the negators 602 on the negator carriage 603 is designed such that the negators 602, when unspooled, maintain a substantially parallel configuration to the base plate 615 so as not to introduce unnecessary torsional forces that can introduce frictional forces that can prevent operation of the driver.

In an exemplary embodiment, once the syringe 618 is loaded and secured such that the face of the syringe plunger 620 is securely held within the syringe plunger receptor 604 and the syringe flange is securely held within the syringe flange receptor 612, the cover 616 may be closed such that the cover opening button 610 is reset. The lever 601 is now at a different angle (not illustrated), rotated about the lever attachment point 613 from its starting position when the pump assembly 103 is not in use. The angle of the lever 601 is dependent on the linkage between the lever 601 and the component(s) that move the negator loading carriage 605, such that the negators 602 can be loaded for pump assembly 103 use.

In an exemplary embodiment, the lever 601 is connected to a belt carriage 609 via a linking arm. The linking arm connects to the lever 601 via a lever connection, such that when connected to the belt carriage 609 on the other end the desired lever 601, an activation force and stroke quantity is achieved. The linking arm is connected to the lever 601 and belt carriage 609 such that the linking arm is parallel to both the lever 601 and belt carriage 609. Further, the belt carriage 609, and thus the distal end of the linking arm, is disposed after the negator loading carriage 605 such that, visually, the negator loading carriage 605 sits between the negator carriage 603 and the belt carriage 609.

In an exemplary embodiment, the belt carriage 609 attaches onto the elevated track 661 of the compact (triple) track rail 611 via a track connection, similarly to the negator carriage 603 and the negator loading carriage 605. The belt carriage 609 is placed on an elevated track (not labeled) of the compact (triple) track rail 611 such that it does not interfere in with the movement of negator carriage 603 and the negator loading carriage 605, which ultimately allows the width of pump assembly 103 to be desirably smaller. The belt carriage 609 has one face equally distanced unidirectional teeth distributed across the length of the face. The opposing face of the belt carriage 609, the smooth inside belt surface, is smooth throughout. The unidirectional direction teeth of belt carriage 609 grips onto opposing unidirectional teeth of the belt 607. The belt carriage 609 grips the entire width of the belt 607. The belt carriage 609 and belt 607 have opposing unidirectional teeth, similar to unidirectional ratchet mechanisms, such that the belt 607 can be moved by the belt carriage 609 in one direction, due to the opposing unidirectional teeth, but be fully unengaged when moved in the opposite direction as a result of the unidirectional teeth releasing (i.e., not gripping) one another. In some exemplary embodiments, the belt carriage 609 grips the full width of the belt 607.

In an exemplary embodiment, similar to the belt carriage 609, the negator loading carriage 605 has opposing unidirectional teeth to the belt 607 and grips the belt 607 in a similar fashion as the belt carriage 609. In some exemplary embodiments, only one side of negator loading carriage 605 grips onto the belt 607. As such, the unidirectional teeth of both the belt carriage 609 and the negator loading carriage 605 are in the same direction.

In an exemplary embodiment, the belt 607 is positioned onto four posts placed on the perimeter corners of the compact (triple) track rail 611, see FIG. 6B, where the belt 607 is positioned at the corners of the compact (triple) track rail 611 as an indication of these posts. These posts are each fitted with a belt roller 606. The belt rollers 606 are made of low-friction material and are allowed to freely rotate around the posts on the perimeter corners of the compact (triple) track rail 611. The belt 607 is placed onto the four posts on the perimeter corners of the compact (triple) track rail 611 such that the smooth face of the belt 671 is in direct contact with all four belt rollers 606 and that the unidirectional teeth 673 of the belt 607 are facing away from the compact (triple) track design 611. In some exemplary embodiments, the belt 607 fits onto all four belt rollers 606 such the belt 607 is snug onto the belt rollers 606 such that it does not fall off when the pump assembly 103 is moved, but not too snug such that the belt 607 cannot easily be rotated around the belt rollers 606. As such, the length of the belt 607 is dependent on the perimeter of the four belt rollers 606. Further, the belt 607 is placed such that base plate 615 cannot interfere with the rotation of the belt 607.

In an exemplary embodiment, when the lever 601 is fully pressed down, the linking arm connected to the belt carriage 609 moves the belt carriage 609 forward. As a result, the unidirectional teeth of the belt carriage 609 grip the opposing unidirectional teeth of the belt 607 thus causing the belt to move. As a result, and simultaneously, the unidirectional teeth of the belt 607 grip the opposing unidirectional teeth of the negator loading carriage 605. As a result, the negator loading carriage 605 is pulled towards the direction of the syringe 618 thus causing the negators 602 to unspool. The negator carriage 603 is limited in motion as a result of the opposing force of the syringe plunger 620 as a result of the administration set (i.e., the subcutaneous needle set 101 or intravenous infusion set 201) being closed/blocked or having high flow due to high flow restrictive administration sets and/or high fluid viscosity.

In an exemplary embodiment, the lever 601 was pressed once, so the negators 602 were unspooled to a certain length. One example may be for delivering a partial dosing. However, this is not the negator 602 unspooling length required to dispense the full 60 ml volume of the specified syringe 618. As the lever 601 was pressed once, three more strokes are required to unspool the negators 602 to the length required to dispense 60 ml volume of the specified syringe 618.

In an exemplary embodiment, the user then returns the lever 601 to the fully opened angle position (not shown), which may be aided by the spring (not shown). Moving the lever 601 in this direction moves the linking arm and the attached belt carriage 609 in the same direction. As a result, the belt carriage 609 unidirectional teeth no longer grip the belt 607 allowing the belt carriage 609 to return to the starting position (not labeled). The lever 601 can be pressed three more times to unspool the negators 602 to the length required to dispense the fully 60 ml volume of the specified syringe 618.

In an exemplary embodiment, during dispensing the lever 601 will be down similar to the “not in use” position. The belt 607 grips and maintains the negator loading carriage 605 in a fixed location. As such, the force of the negators 602 attempting to re-spool causes the negator carriage 603 and the syringe plunger receptor 604 to move towards the syringe 618. As a result, the force of the negators 602 acts upon the syringe plunger 620 causing the syringe plunger 620 to dispense the contents of the syringe 618 once the drug path is allowed to flow. In some exemplary embodiments, the components are distanced such that the total allowable volume of the syringe 618 is dispensed.

In an exemplary embodiment, once the contents of the syringe 618 are fully dispensed, the belt release clip 608 may be pressed to push unidirectional teeth of the belt 607 out-of-line with the opposing unidirectional teeth of the negator loading carriage 605 such that negator carriage 603, syringe plunger receptor 604 and negator loading carriage 605 can freely be pushed back towards the starting position such that the syringe 618 can easily be removed and the pump assembly 103 can be used again. The belt release clip 608 may be pressed while dispensing the syringe 618 as deemed necessary by the user, thus stopping the infusion. Doing so releases the belt 607 from the negator loading carriage 605, which may cause the negator loading carriage 605 to travel back towards the negator carriage 603 as a result of the syringe plunger 620 limiting the motion of negator carriage 603 for reasons explained previously. As a result, part damage or louds unpleasant noises may occur. To reduce this, a cushioned brake may be placed between negator carriage 603 and the negator loading carriage 605. The cushioned brake does not interfere with any motion.

In an exemplary embodiment, the belt grips 614 placed on the base plate 615 act as mechanicals supports for pressing the belt release clip 608 and cover opening button 610 and as such are appropriately placed to achieve said support.

In an exemplary embodiment, the lever 601 and cover 616 can be closed post-pump use, thus resetting the cover opening button 610.

Table 1 below shows exemplary required lengths of series tubing 110 at a specified inner diameter required to calibrate the flow dials on the variable flow rate controller 107 (from FIG. 1A). The infusion fluid of Table 1 is specific to 20% immunoglobulins (i.e., Hizentra®) dispensed with a constant pressure source of 13.5 psi and whose viscosities may range from 13-17 centipoises. Given the needle 140 length (0.98″-1.05″) and inner diameter (0.0104″-0.0135″) and needle tubing 110 length (18″-26″) and inner diameter (0.038″-0.042″) remain constant between subcutaneous administration sets 101 only the length of the series tubing 110 must be changed, once an inner diameter is selected, to calibrate the variable flow rate controller 107, such that the maximum flow rate in the provided example is 60 ml/hr per the number of needles 140 within a needle set 101. Once a series tubing 110 inner diameter is selected, the series tubing 110 length required to maintain the flow dials on the variable flow rate controller 107 within calibration can be determined. Simply, the series tubing 110 length is determined such that flow rate (of the variables in the provided example) dispensed from each needle within the needle set 101 is 60 ml/hr when the variable flow rate controller 107 is set to the maximum position. Of course, a skilled artisan will appreciate that specific flow rates can be achieved with numerous other combinations of tubing and needle lengths and diameters besides the examples shown in Table 1 below.

TABLE 1

| Subcutaneous | | Series Tube | Maximum |
|---|---|---|---|
| Administration | Series Tube | Inside | Flow Rate |
| Set Type | Length (in) | Diameter (in) | (mL/hr) |
| 1-Needle | 8.5-12 | 0.015-0.020 | 60 |
| 2-Needle | 11.5-17 | 0.020-0.025 | 120 |
| 3-Needle | 7.5-12 | 0.020-0.025 | 180 |
| 4-Needle | 5.5-8.5 | 0.020-0.025 | 240 |

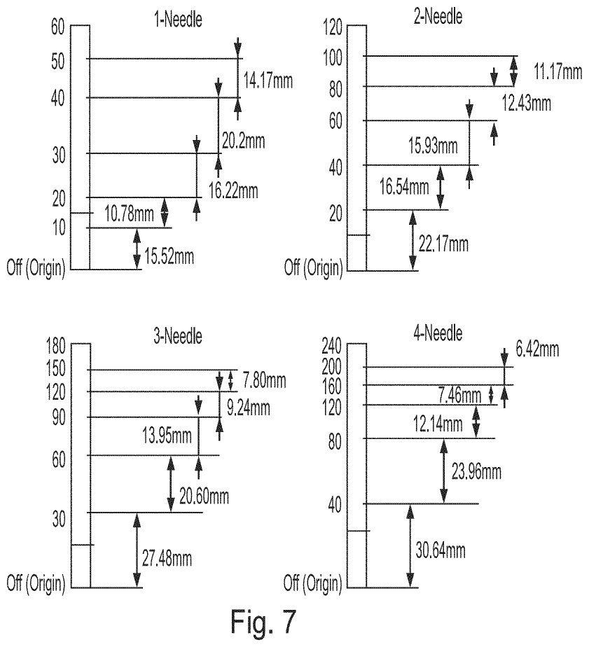

FIG. 7. shows the calibrated flow dials of the variable flow rate controller 107 for 1, 2, 3 and 4-needle administration (needle) set 101 for flow rates of 10, 20, 30, 40, 50 and 60 ml/hr for infusion fluid and infusion and administration set parameters provided in the example presented in Table 1. For the desired flow rate to achieve the lengths and diameters of fluid-pathed components (i.e., the needle, needle tubing, series tubing) must be known. These values may be determined experimentally or optically. Optical methods of determination include direct measurements of inner diameters using optical tools such as compound microscopes. Experimentally, the flow rate can be measured fluidically or using air measurement methods such as flow meter systems. Given the length of the fluid-pathed component and the experimentally measured flow rate the inner diameter of the fluid-pathed component can be calculated using the Hagen-Poiseuille equation (HPE).

The HPE can be used to determine the flow rate of a fluid, with a viscosity, given the length and radius of fluid-pathed components (i.e., the needle tubing) within the administration set, and the differential pressure between the pressure source (i.e., the infusion driver) and the patient's infusion anatomic site. The HPE may be rewritten to solve for any of its variables, including inner diameter of the fluid pathed components. To use the HPE, the following assumptions must be met: the fluid is incompressible, Newtonian, is not accelerating within the administration set, is in laminar flow through the fluid-pathed components of the administration set that maintain a constant circular cross-sectional area and has a length that is substantially larger than its diameter.

Given the above, the HPE can be written as equation (1) below:

$$Q = \frac{Δ p π {R}^{4}}{8 L μ} \qquad ( 1 )$$

where:
Q is the volumetric flow rate of the infusion fluid;
Δp is the differential pressure between the pressure source and the patient's infusion anatomic site;
R is the radius of the fluid-pathed component;
L is the length of the fluid-pathed component; and
μ is the dynamic viscosity of the infusion fluid.

The HPE in combination with a total flow equation (TFE) can be used to determine the flow rate of fluid-pathed flow rate-impacting components within the administration set and the flow rate of the entire administration set.

The flow rates (Q) of each fluid-pathed component must be combined to determine the total flow rate of the administration set. This may be done using the TFE (2) below:

$${Q}_{Total Flow Rate} = \frac{( {Q}_{Series Tubing} ) ( {Q}_{Needle and Needle Tubing} )}{( {Q}_{Series Tubing} + {Q}_{Needle and Needle Tubing} )} \qquad ( 2 )$$

where:

Q_{Total Flow Rate} is the total flow rate of the administration set;

Q_{Series Tubing} is the flow rate of the series tubing 110; and

Q_{Needle and Needle Tubing} is the flow rate of the needle and needle tubing combined.

Knowing the total flow rate of the administration set and of the needle 140 and needle tubing 110 the TPE can be rewritten and solved for the flow rate of the series tubing 110. Given the inner diameter of the series tubing 110 and flow rate the HPE can be used to determine length of the series tubing 110 required to calibrate the administration set such that each needle dispenses a maximum flow rate of 60 ml/hr.

A similar example may be provided for intravenous infusion sets 201 in which the series tubing 210 is at a set inner (0.01780″-0.01820″) whose lengths may be adjusted such that the variable flow rate controller 207 when dispensing an infusion fluid of low viscosity about 1 centipoise (i.e., antibiotics such as Vancomycin®) dispensed with a constant pressure source of 13.5 psi has a maximum flow rate of 300 ml/hr. The flow dials of the variable flow rate controller 207 may be calibrated from 5-300 ml/hr.

### Other Infusion Driver Mechanisms

It is noted that the driver may include various mechanisms. Some embodiments of the infusion driver may include other mechanisms to load the negators. This loading of the negators can be achieved by using the belt carriage 609 of FIGS. 6B and 6D. However, in other embodiments, the loading of the negators may be achieved by use of a ratchet and track loading mechanism constructed according to the principles and embodiments of the invention as described below. The ratchet and track embodiments has numerous advantages including reducing the number of parts including, e.g., the need for the belt carriage 609, belt 607, and/or belt rollers 606, which greatly reduces the size of the driver, increases ease of assembly increases reliability, lowers costs, and provides a more robust design. Other advantages include increasing ease of use, e.g., by using a lever to load the driver, reducing runaway motion in normal use, providing for multi-dosing capability with different dosing amounts for each lever use (e.g., 10 or 15 ml per actuation of the lever), preventing injury and reducing the risk of pinching since the lever of the infusion driver is not part of the cover and preventing breakage (e.g., by not requiring a velocity limiter or active braking system when in use).

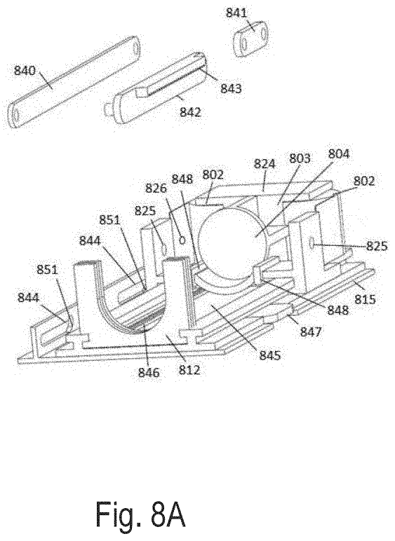

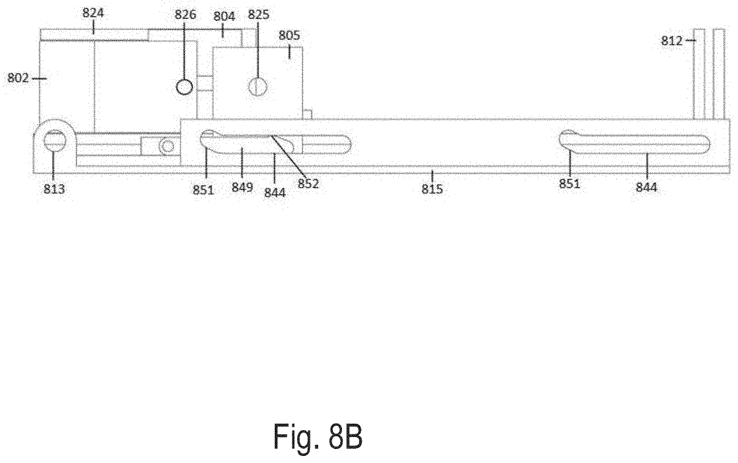

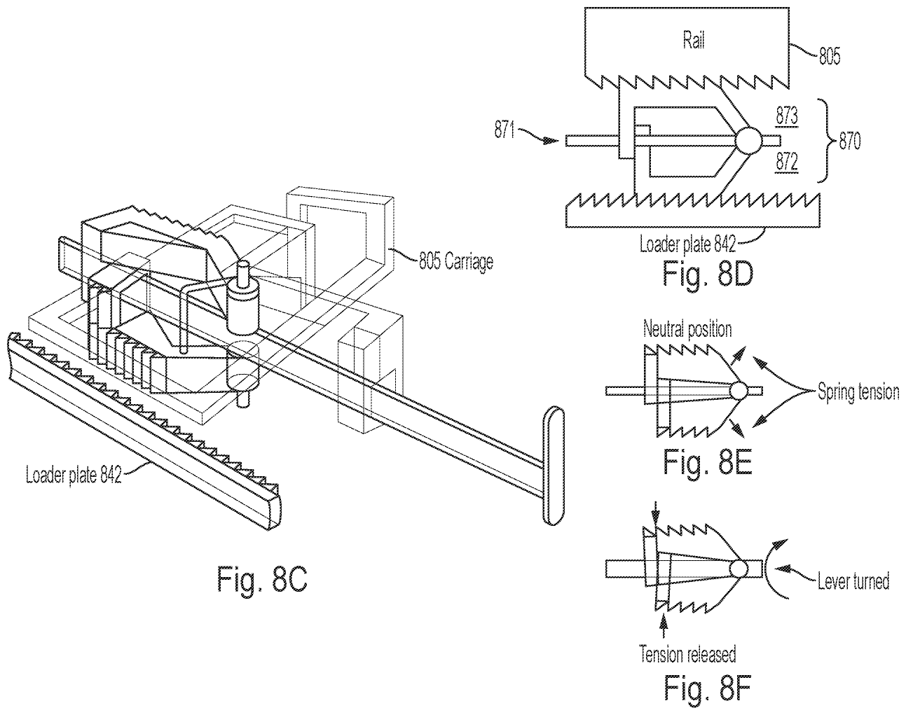

In FIGS. 8A-8F, the belt carriage 609 (shown in FIGS. 6B and 6D) may be replaced with a unidirectional toothed clip 842 similarly connected to the linking arm 840 such that the unidirectional teeth 843 of the unidirectional toothed clip 842 are equally distanced and disposed onto one face of the unidirectional toothed clip 842. The unidirectional toothed clip 842 is aligned with the longitudinal centerline of the fluid reservoir and parallel with moving components which also eliminates torsional force and thus eliminates loss of pressure performance on the syringe plunger. When connected to the linking arm 840, the teeth 843 of the unidirectional toothed clip 842 point toward the base plate 815. Further, the unidirectional toothed clip 842 sits in a groove 844 such that when moved, it is parallel to the motion the negator loading carriage 805.

In one exemplary embodiment of the driver mechanism, unidirectional teeth of the negator loading carriage 605 may be replaced with an opposing unidirectional toothed clip 849. The opposing teeth 852 of the opposing unidirectional toothed clip 849 of the negator loading carriage 805 is faced opposing and in line with the teeth of the unidirectional toothed clip 842 connected to the linking arm 840 such that when moved in opposing directions the opposing unidirectional teeth 852 of the opposing unidirectional toothed clip 849 of the negator loading carriage 805 is gripped, and subsequently pulled, by teeth 843 of the unidirectional toothed clip 842 connected to the linking arm 840. As a result, when the lever 601 (shown in FIG. 6A) is pressed the negators 802 begin to unspool.

At the proximal end of the groove 844 is a curved feature 851 that lifts the unidirectional toothed clip 842 upwards such that it may clear the opposing teeth 852 of the opposing unidirectional toothed clip 849 of the negator loading carriage 805 when the lever 601 is at the fully opened angled. When the lever 601 is pressed the unidirectional toothed clip 842 interferes and grips the opposing teeth 852 of the opposing unidirectional toothed clip 849 of the negator loading carriage 805 as the unidirectional toothed clip 842 travels to the distal end of the groove 844. This mechanism can be used to achieve the same lever 601 activation force and stroke quantity as previously mentioned. As the unidirectional toothed clip 842 advances, it grips and pulls the opposing teeth 852 of the opposing unidirectional toothed clip 849 of the negator loading carriage 805 the negators 802 unspool a certain length as previously mentioned. This consistent force and stroke quantity limits the dosing amount to 10 ml or 15 ml for each actuation of the lever 601. However, in other embodiments with larger reservoir sizes and/or track loading lengths different dosing amounts may be achieved.

To prevent the negators' 802 distal ends connected to the negator loading carriage 805 from spooling backwards to the negator carriage 803, a unidirectional set of teeth 848 may be used on the negator loading carriage 805. The compact (triple) track rail 611 (shown in FIGS. 6B-6D) may be replaced with a toothed track 845 with a set of opposing unidirectional teeth 846 that oppositely face the unidirectional set of teeth 848 on the negator loading carriage 805 such that unidirectional set of teeth 848 on the negator loading carriage 805 are gripped by the opposing unidirectional teeth 846 of the toothed track 845, thus preventing the negators' 802 distal ends connected to the negator loading carriage 805 from spooling backwards to the negator carriage 803.

To reset the position of the negator loading carriage 805, once the device is used, a track pushing clip 847 that can be pressed to push the opposing unidirectional teeth 846 of the toothed track 845 out-of-phase with the unidirectional set of teeth 848 on the negator loading carriage 805 such that the negator loading carriage 805, negators 802, and negator carriage 803 can be freely moved along the toothed track 845 and can be prepared for the next device use. Further, the track pushing clip 847 is positioned such that when pushed, the toothed track 845 is pushed orthogonally to the direction of carriage motion of the toothed track 845. In one exemplary embodiment, a cushioned brake may be used to cushion the negator loading carriage 805 and negator carriage 803 contact in the event the tracking pushing clip 847 was activated during device use, as previously mentioned.

As shown in FIGS. 8C-8F, in one exemplary embodiment, a negator loading mechanism and a negator loading carriage reset mechanism are combined with shared components thus reducing component quantity and overall driver size. This is achieved using a turnable shaft and key scissor mechanism 870 placed in between the unidirectional toothed clip 842 (of the loader plate) and negator loading carriage 805. As shown in FIG. 8E, when the shaft 871 is in the neutral position, the loader plate key 872 of the key scissor mechanism 870 mates with the unidirectional teeth 843 of the unidirectional toothed clip 842. Simultaneously, the negator loading carriage key 873 of the key scissor mechanism 870 mates with the unidirectional set of teeth 848 of the negator loading carriage 805. Thus, the driver loading mechanism functions as described above. As shown in FIG. 8F, when the shaft 871 is rotated 90 degrees the key scissor mechanism 870 has its tension released, and thus the loader plate key 872 and the negator loading carriage key 873 is freed from the unidirectional teeth 843 of the unidirectional toothed clip 842 and the unidirectional set of teeth 848 of the negator loading carriage 805, respectively, thus removing all forces from the driver and enabling the user to reset the negator loading mechanism.

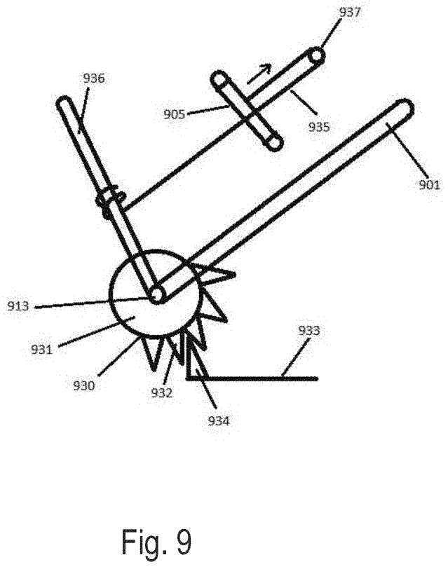

In FIG. 9, the belt carriage 609 of FIGS. 6B and 6D is replaced with a unidirectional toothed clip. In an exemplary embodiment, the lever 901 is combined with a toothed wheel 930 placed at around the lever's 901 point of rotation around the lever attachment point 913 in which the connection is formed via a one direction double spool arrangement 931. The one direction double spool arrangement 931 enables the lever 901 to be operated in the upward or downward direction in which the teeth 932 of the toothed wheel 930 move only in one direction. A ratchet component 933 may be used with ratchet teeth 934 that opposingly face the teeth 932 of the toothed wheel 930. The ratchet component 933 is connected to a shaft 936 such that when the lever 901 is pressed the cable 935 is spooled around the shaft 936. The shaft 936 is placed at the lever 901 point of rotation about the lever attachment point 913 such that the long axis of the shaft 936 is orthogonal to the face of the toothed wheel 930. The shaft 936 further connects to the negator loading carriage 905 via a pulley system 937 and an extension of the cable 935. As the lever 901 is pressed and the cable 935 is spooled the negator loading carriage 905 unspools the negators. Post-device use of the ratchet component 933 can be reset automatically or via a ratchet release button that functions similarly to the track pushing clip 947 and the belt release clip 909, as mentioned previously.

Although certain exemplary embodiments and implementations have been described herein, other embodiments and modifications will be apparent from this description. Accordingly, the inventive concepts are not limited to such embodiments, but rather to the broader scope of the appended claims and various obvious modifications and equivalent arrangements as would be apparent to a person of ordinary skill in the art.

The claimed invention is:

1. An assembly to deliver a fluid into a patient's anatomic space, the assembly comprising:
a body having an opening to receive a needle to penetrate into a patient's anatomic space, and
extensions hinged to the body and movable between a closed, compact position and an expanded, open position without any significant biasing force tending to force the extensions into either position, each extension having an outer periphery at least partially surrounding an interior surface having a locking structure to mate with the other extension and lock the extensions together in the closed, compact position.

2. The assembly of claim 1, wherein the interior surfaces of the extensions further comprise:
a guide to prevent misalignment during movement of the extensions to the closed, compact position.

3. The assembly of claim 2, wherein the guide comprises a guiding mechanism having a curved protrusion extending from the interior surface of one of the extensions.

4. The assembly of claim 1, wherein the locking structure is configured to provide sensory feedback to a user to indicate the extensions are in the closed, compact position.

5. The assembly of claim 1, wherein the body comprises a hub and the extensions comprise a pair of wings extending from the hub, with the locking structure on one wing comprising a projection and the locking structure on the other wing comprising a recess to receive the projection.

6. The assembly of claim 1, wherein at least one of the extensions comprises:
a raised surface including a plurality of substantially smooth, spaced projections to increase the surface area of the interior surface and create a diffuse patient contact surface for contacting the patient without causing substantial pain or irritation.

7. The assembly of claim 5, further comprising hinges connecting the wings to the hub, the hinges being in a neutral and substantially un-compressed state when the wings are in the open, expanded position and wings hinges being disposed no more than about 10 degrees from horizontal when the wings are in the open, expanded position.

8. The assembly of claim 7, wherein each hinge is less than about 0.3 mm thick and about three or less pounds of force is required to lock the wings in the compact, closed position and about six or more pounds of force is required to unlock the wings.

9. The assembly of claim 1, further, comprising:
a needle in fluid communication with the body and having an end configured to penetrate into a patient's anatomic space to deliver fluid thereto;
a needle protector surrounding the needle; and
a groove disposed in at least one of the extensions to guide and maintain the needle protector in the groove before use.

10. The assembly of claim 1, further comprising a ball-and-pivot mechanism to receive a portion of a fluid delivery needle and allow rotation by the needle to reduce forces transmitted to the needle during use of the assembly.

11. An assembly to deliver a fluid into a patient's anatomic space, the assembly comprising:
a needle to penetrate into a patient's anatomic space;
a needle protector surrounding the needle;
a body having a longitudinal axis and an opening to receive the needle; extensions hinged to the body and movable between a closed, compact position and an expanded, open position, each extension having an outer periphery at least partially surrounding an interior surface having a locking structure to mate with the other extension and lock the extensions together in the closed, compact position; and
a recess disposed in at least one of the extensions to guide and maintain the needle protector in a substantially 90 degree orientation relative to the longitudinal axis of the body.

12. A method for delivering a fluid into a patient's anatomic space through a needle having a needle protector and being supported by a butterfly assembly having a base and a pair of wings extending from the base, with a guiding channel supporting the needle protector in a substantially 90 degree relative to the longitudinal axis of the base, the wings being movable between a compact, closed position surrounding the needle protector and an open, expanded position exposing the needle for insertion into the patient's anatomic space, the method comprising:
moving the butterfly assembly into the open, expanded position exposing the needle and the needle protector;
removing the needle protector;
inserting the needle into a patient's anatomic space;
and
pressing the pair of wings against the patient without any significant biasing force tending to force wings into the closed position.

13. The method of claim 12, further comprising the step of:
delivering a therapeutic treatment fluid into the patient's anatomic space via the needle.

14. The method of claim 12, wherein the step of pressing the pair of wings against the patient comprises presenting a plurality of spaced, substantially smooth raised surfaces on the wings to the patient to increase the surface area of wings and create a diffuse patient contact surface for contacting the patient without causing substantial pain or irritation.

15. The method of claim 12, wherein the step of moving the butterfly assembly into the open, expanded position comprises moving the wings about a hinge less than about 0.3 mm thick to the open, expanded position before pressing the wings against the patient.

16. The method of claim 13, further comprising the step of:
withdrawing the needle from the patient;
guiding the wings into the closed position with a curved protrusion extending from an interior surface of one of the wings; and
locking the wings in the closed, compact position with a locking mechanism disposed on interior surfaces of the wings.

17. The method of claim 16, further comprising the step of:
providing sensory feedback to indicate the wings are in the closed, compact position.

18. The method of claim 12, wherein about three or less pounds of force is required to lock the wings in the compact, closed position and about six or more pounds of force is required to unlock the wings.

19. An infusion assembly for delivery of an infusion fluid into a patient's anatomic space at a substantially constant pressure, the infusion assembly comprising:
a reservoir for the infusion fluid; and
a pressurizing mechanism to deliver the infusion fluid from the reservoir into the patient's anatomic space at a substantially constant pressure, the pressurizing mechanism including a substantially constant force spring mechanism in contact with a pressurizer and an actuator to gradually load the substantially constant force spring mechanism.

20. The infusion assembly of claim 18, wherein the substantially constant force spring mechanism comprises one or two springs, the pressurizer comprises a plunger, and the actuator comprises a lever and a ratcheting mechanism.
